(19) United States
(12) Patent Application Publication
(10) Pub. No.: US 2010/0173793 A1
(43) Pub. Date: Jul. 8, 2010

(54) PEPTIDE / PROTEIN IDENTIFICATION USING PHOTOREACTIVE CARRIERS FOR THE IMMOBILISATION OF THE LIGANDS

(75) Inventors: Suzanne Jane Dilly, Overseal (GB); Paul Christopher Taylor, Holmfirth (GB); Andrew James Clark, West Midlands (GB); Andrew Marsh, West Midlands (GB); Richard Napier, Warwickshire (GB); Andrew Thompson, Warwickshire (GB)

Correspondence Address: BARNES & THORNBURG LLP, P.O. BOX 2786, CHICAGO, IL, 60690-2786, US

(73) Assignee: UNIVERSITY OF WARWICK, Coventry (GB)

(21) Appl. No.: 12/063,803

(22) PCT Filed: Aug. 17, 2006
(86) PCT No.: PCT/GB06/03089

§ 371 Date: May 27, 2008

(30) Foreign Application Priority Data
Aug. 17, 2005 (GB) 0516870.3
Jan. 31, 2006 (GB) 0601964.0
May 9, 2006 (GB) 0609175.5

(51) Int. Cl. C40B 30/04; C07D 229/02; C08G 63/91; C08G 69/48; B01L 3/00; C40B 40/10

(52) U.S. Cl. 506/9; 548/960; 525/418; 525/420; 422/99; 506/18

(57) ABSTRACT

The invention provides a method of identifying a peptide or protein capable of binding a ligand which comprises: (i) providing a support, the support comprising a photoreactive group; (ii) reacting the photoreactive group with a ligand to attach the ligand to the support and produce a supported ligand; (iii) providing an expression library comprising a plurality of members, each member expressing a different peptide or protein; (iv) screening the expression library to identify one or more peptides or proteins which bind to the ligand; (v) isolating the member or each member of the library which expresses a peptide or protein which binds to the ligand; and (vi) identifying the peptide or protein which binds to the ligand. Supported photo-reactive compounds are disclosed comprising a photo-reactive group attached to a support via a spacer and a dendritic group, the dendritic group comprising attached thereto, optionally via a spacer, at least one further photo-reactive group and/or a second functional group. Compounds comprising photo-reactive groups attached to a support and a protein resistant group attached to the support are also provided, together with kits for carrying out the method of the invention.

The invention relates to methods for identifying peptides or proteins capable of binding ligands, to supported photo-reactive compounds for use in such methods and kits for use in such methods. The methods may be used to immobilise a wide variety of small molecules.

The use of surface-display vectors for displaying polypeptides on the surface of, for example, bacteriophage, bacteria or yeast has been used for a number of years for the manipulation of ligands such as enzymes, antibodies and peptides. These methods are reviewed in, for example, the article by Benhar I. (Biotechnology Advances (2001), Vol. 19, pages 1-33). Phage display is based on expressing recombinant proteins or peptides fused to a phage coat protein. Bacterial display is based on expressing recombinant proteins fused to sorting signals that direct their incorporation onto the cell surface. The phage or bacteria expressing the recombinant peptides or proteins are used, for example, to screen for their ability to bind different ligands.

One problem associated with such assays is that it is often useful to have the ligand bound to a solid support in order to allow the bacteria or phage expressing the peptide or proteins to be isolated from solution.

Conventionally, potential ligands of a peptide or protein are attached to a support by means of a chemical reaction designed to cause the support to bind to a specific part of the molecule. For example, 3-indole acetic acid may be attached to hydroxy or amino functionalised supports by esterifying the molecule, followed by a Mitsunobu reaction to couple the protected indole acetic acid to the support. This reaction specifically targets a hydroxyl group on the compound and results in the compound being presented to the surrounding solution in a regio-specific manner. Similarly, other compounds may be reacted with supports, with the prior knowledge of the structure of the ligand to be attached to the support. This requires the advanced knowledge of the structure of the ligand, the knowledge of often complex chemistry to attach the ligand to the support, and often results in the ligand being presented to the surrounding solution in such a way that parts of the ligand which are important for binding to, for example, a peptide, are not presented to the peptide correctly. This means that that binding to the peptide may not occur, thus leading to false negative results. The inventors have therefore recognised that there is a need to be able to produce a controllable and relatively simple method of attaching ligands to a substrate, without the need for complex chemical reactions or the prior knowledge of the structure of the ligand, which can be used in combination with an expression system, such as a phage display library, and which is readily adaptable to automation to allow the screening of large numbers of different compounds or peptides.

Kanoh N., et al. (Angew. Chem. Int. Ed. (2003), Vol. 42, pages 5584-5587) discloses a protocol for immobilising products on glass slides using a photoaffinity reaction. The protocol utilises photo-reactive group containing molecules attached to glass slides. Solutions containing small molecules are washed over the slide and exposed to light. This results in the small molecule reacting with the photo-reactive group and results in the binding of the molecule to the slide. The bound molecules may be used to detect the interactions between proteins and small molecules. The use of a diazirine is exemplified.

The assay disclosed in Kanoh is demonstrated for the binding of proteins and antibodies in solution. There is no suggestion that such a system would allow receptors expressed in an expression library would be able to bind ligands attached to the photo-reactive group.

The same group immobilised molecules such as rhodamine B via supported photo-reactive groups (Kanoh, et al., Angew. Chem. Int. Ed. (2005), 44: 3559-3562). Proteins binding the molecules were identified using antibodies requiring the prior knowledge of the protein binding the molecule. MALDI-TOF MS was used by the authors to obtain a protein mass fingerprint. However, this required using database matching to try to identify the protein via its mass. The invention provided by the Applicants does not need this information.

WO 2004/090540 is by the same group. It discloses using photo-reactive groups on supports and evaporating to dryness the reaction mixture prior to photo-irradiation to immobilise low molecule weight compounds. In contrast to this the current invention does not require evaporation to dryness.

Moreover, Bradner, et al. (Chemistry and Biology (2006), 13: 493) tried the techniques used by Kanoh, et al. This provided unacceptable numbers of false positives as judged by secondary binding assays using surface plasmon resonance. This experience lead the authors of the Bradner paper to pursue different techniques that would allow immobilisation of functional groups.

WO 2004/088316, US 2005/0170427A and WO 2004/002995 confirm that immobilising molecules such as nucleic acids using photo-reactive groups is known in the art.

Uttamachani M. et al. (Curr. Opin. Chem. Biol. (2005), Vol. 9, pages 4-13) discusses small molecule microarrays and their use in combinatorial chemistry. It discusses library design and synthesis using sophisticated, complicated chemistry to immobilise compounds onto substrates. The paper reports attempts to use PNA tags to remove some of the complexity of producing libraries. It also uses photoactivated supports to immobilise small molecules and screen them against proteins, such as enzymes. The assay disclosed is aimed at finding enzymes. This requires the need for a metabolisable substrate. This is in contrast to the assay developed by the inventors to find ligands binding to proteins.

The inventors have identified that it is possible to utilise photo-reactive groups to bind ligands to a support. The bound ligand can then be used to screen expression libraries, expressing peptides or proteins, for peptides or proteins that bind to the ligand. This system has a number of advantages:

(i) The use of a photo-reactive group allows ligands to be bound to a support without need for knowledge of the chemical structure of the ligand. It requires no prior knowledge of the binding position of the ligand to the receptor. Conventional chemical synthesis of supported ligands is complicated, requiring large amounts of chemical skill and may bind the support to the functional binding part of the ligand, thus inhibiting ligand-peptide interactions. The use of the photo-reactive group requires less chemical skill and allows the ligand to be displayed in a number of different orientations to maximise the probability of biological interaction with the peptide or protein.

(ii) The possibility of a ligand being bound by a support can be maximised by using two or more different photo-reactive groups attached to the support. This can also be used to maximise the number of positions on the ligand that the support attaches to, thus increasing the number of different orientations in which the ligand is presented on the surface of the support for screening for binding to the peptides or proteins.

The use of an expression library allows peptides or proteins that bind to the ligands to be rapidly identified and the nucleic acid sequence encoding that peptide easily determined. This is especially useful for the identification of unknown receptors for a ligand. The system identified by the inventors allows the rapid identification of new receptors and the nucleotide sequence of those receptors without needing prior knowledge of the ligand-receptor interaction or their mode of interaction. This may be used to identify receptors that drugs or drug candidates bind to.

The system identified by the inventors can be optimised to reduce non-specific binding to the expression library, increase the accessibility of the ligand to the library, and to allow the system to be used as a bench-based or automated assay.

The invention provides a method of identifying a peptide or protein capable of binding a ligand which comprises:
(i) providing a support, the support comprising a photo-reactive group;
(ii) reacting the photo-reactive group with a ligand to attach the ligand to the support and produce a supported ligand;
(iii) providing an expression library comprising a plurality of members, each member expressing a different peptide or protein;
(iv) screening the expression library to identify one or more peptides or proteins which bind to the ligand;
(v) isolating the or each member of the library which expresses a peptide or protein which binds to the ligand; and
(vi) identifying the peptide or protein which binds to the ligand.

In an especially preferred embodiment, two or more different supports are used, each support having a different photoreactive group. Alternatively, two or more different photoreactive groups may be provided on the same support. This allows the range of ligands to which the photoreactive groups can bind to be increased and/or to increase the number of ways in which the ligand is presented to the library because of differences in the parts of the ligand to which the photoreactive groups bind.

The term peptide or protein is intended to mean a sequence of amino acids held together by a peptide bond. Preferably “peptide” means that the peptide contains less than 50, less than 45, especially less than 40, less than 30, less than 20, preferably more than 2 amino acids held together by peptide bonds.

The ligand may be any compound which potentially could bind to a peptide or protein. This includes, but is not limited to, drugs, drug candidates, hormones, peptides, carbohydrates. Preferably the ligand is not a protein or a nucleic acid molecule, but not excluding peptides. Preferably the ligand is not metabolised upon binding the peptide or protein.

The peptide or protein is preferably not an enzyme. The peptide or protein is preferably a receptor or a fragment thereof.

The peptide or protein identified in step (vi) is preferably:

(i) sequenced to establish the amino acid sequence of the peptide and protein; or
(ii) a portion of the member of the expression library encoding the peptide or protein is sequenced to identify a nucleotide sequence encoding the peptide or protein.

This allows, for example, the protein to be identified with a particular phenotype. The ability to identify the nucleotide sequence is especially preferred as sequencing is relatively rapid and can then be readily searched and analysed.

Once the amino acid sequence or the nucleotide sequence encoding the peptide or protein is known, this can be compared with other databases to obtain bioinformatic information about the protein or peptide, utilised to produce a probe or a primer to isolate, for example, the full gene encoding the protein or peptide, or otherwise manipulated to allow the further characterisation of the protein or peptide.

Potentially, any expression library capable of expressing a peptide or a protein so that it can be assayed against the supported ligand may be used in the method of the invention. The libraries contain nucleic acid sequences encoding the peptide or proteins. Preferably, the library is a surface display library. It may be a prokaryotic or eukaryotic library. Such surface display libraries are known in the art. Preferably the library is a cell-based library. Preferably, the display library is selected from a phage display library, a bacterial cell surface display library, a yeast cell surface display library and a baculovirus insect expression library. Virus display libraries, such as baculovirus or phage display libraries are especially amenable to high throughput assays.

The article Benhar I. (Supra) and the article by Wernérus H. and Stahl S. (Biotechnol. Appl. Biochem. (2004), Vol. 40, pages 209-228) review, for example, phage and bacterial expression systems which are suitable for use in the method of the invention. Phage display libraries tend to be based on expressing the recombinant proteins or peptides fused to a phage coat protein. The phage used may be a filamentous phage display library which is based on cloning DNA fragments encoding variants of peptides and proteins or fragments thereof into a phage genome, fused to the gene encoding preferably one of the phage coat proteins. Upon expression, the coat protein fusion is incorporated into new phage particles that are assembled into the periplasmic space of a bacterium infected by the phage. Expression of gene fusion product and its subsequent incorporation into the mature phage coat results in the protein or peptide being presented on the phage surface, whilst its genetic material resides within the phage particle.

Phage that display a relevant ligand are retained on the surface of the supported ligand and may be recovered from the surface of the support, used to reinfect bacteria and may be reproduced for further enrichment for eventual analysis.

Suitable bacteriophage include M13 (for example coat proteins pIII (minor), pVI, pVIII (major), pVII/pIX), λ (for example fused to the D (head protein) or pV (tail protein), P4 (e.g. Psu capsid protein), T7 (e.g. 10B capsid protein), T4 (Hoc capsid protein, Soc capsid protein or internal protein III) or MS2 (coat protein).

The expression library may be contained within the bacterium cell surface display library. A number of different libraries have been disclosed in the prior art (see Benhar (Supra)). The library may be in a gram negative bacteria (such as E. coli, Salmonella, Caulobacter) or gram-positive bacteria such as Streptococcus, Staphylococcus or Bacillus anthracis). Such display libraries are known in the art.

Alternatively, the expression library may be a eukaryotic library, such as yeast, insect or mammalian library. Examples of the expression of heterologous proteins on the outer surface of yeast and mammalian cells are reviewed, for example, in the article by Schreuder, et al. (Vaccine (1991), Vol. 14, pages 383-388). Commercially available mammalian expression vectors designed to target recombinant proteins to the surface of mammalian cells are known in the art and are commercially available. For example, Invitrogen Limited produce a commercially available expression vector (pDisplay™) that allow proteins of interest to be targeted and anchored to the cell surface by cloning the gene of interest in frame with an N-terminal cell surface targeting signal and a C-terminal transmembrane anchoring domain derived from platelet-derived growth factor receptor.

Baculovirus cell surface display libraries are also known in the art, as disclosed for example in the article by Ernst W. (Nucleic Acids Research (1998), Vol. 26(7), pages 1718-1723).

The library preferably contains cDNA or fragments of cDNA, which are expressed in the expression library. The cDNA is preferably exogenous to the host cell. Expression libraries containing cDNAs from, for example, eukaryotic organisms such as plants, or mammals, such as humans, are known in the art. The advantage of using a phage display library containing cDNA as the source of the peptides is that the system allows the identification of previously unknown proteins or peptides that bind to the ligand. As indicated previously, the combination of the ligand bound to the protein, the protein being within a display library, allows the rapid identification of the nucleic acid sequence associated with that protein, and therefore the rapid identification and characterisation of the protein or peptide bound to the ligand. Techniques, such as PCR may be used to selectively amplify the sequence of nucleic acid encoding the peptide or protein.

Alternatively, the library may be a random or non-random peptide library.

Tabuchi I., et al. (BMC Biotechnology (2004), Vol. 4, page 19) discloses a method of making random peptide libraries for evolutionary protein engineering based on a combinatorial DNA synthesis method.

Pinilla C., et al. (Nature Medicine (2003), Vol. 9, pages 118-122) reviews the development of synthetic combinatorial methods and applications of mixture-based combinatorial libraries. This includes the production of libraries of peptides where, in each separate library, one or more amino acids is predefined at a specific position in the peptide, with the remaining positions of the peptide having different amino acids. The predefined amino acid may be at different positions in different libraries. This position scanning format allows, for example, extensive structural information for the binding of peptides with a binding compound to be gathered.

U.S. Pat. No. 6,479,641 discloses the production of libraries to screen for binding moieties, such as parvovirus B19 binding peptides. It discloses the production of a candidate binding domain template which is used as the basis for peptides to be displayed in the library. The binding domain template may be based on knowledge of a known interaction between a known protein or peptide and a compound of interest.

The peptide may be structured, for example, as described in U.S. Pat. No. 6,479,641, as opposed to an unstructured, linear peptide.

Advantageously, the use of the library allows the nucleic acid sequence encoding the peptide or protein found to bind to the ligand to be easily amplified, by, for example, PCR. This allows relatively low concentrations of ligand to be used if necessary.

Photo-reactive groups typically use chemical moieties that on irradiation produce a reactive intermediate that will covalently bond to the ligand. Preferably the irradiation uses visible or ultra-violet light, such as approximately 700-400 nm wavelength for visible light and about 400 nm to 4 nm. The ultra-violet light may be UV-A (320-400 nm), UV-B (280-320 nm) or UV-C (below 320 nm).

The photo-reactive group is activated by exposure to light and reacts with the ligand to form the supported ligand.

Preferably, the photo-reactive group is substantially unreactive under visible light. It may be selectively activatable using ultra-violet light. This allows the photoreactive group to be more easily handled in the laboratory, for example, without the use of light-excluding bags or darkrooms.

Alternatively, benzophenones may be advantageously used.

Benzophenones may react in daylight. They have advantages over photoreactive groups, such as diazirines because they do not decompose upon exposure to light. Diazirines decompose on exposure to light. Benzophenones, however, become excited upon exposure to light but do not decompose. If no ligand is present, they simply return to their original ground state after exposure to light.

Preferably, the benzophenone is substituted or non-substituted. Preferably the substitution is selected from hydroxy, amino, alkoxy, halogen (such as fluorine, bromine, chlorine or iodine), carboxy, carboxyamine, carboxyl, cyano or nitro. Preferably the benzophenone is 4-substituted. Most preferably the benzophenone is an amino benzophenone, especially 4-amino benzophenone.

Preferably, the spacer comprises at least two or more linked carbon atoms separating the photo-reactive group from the surface or support. More preferably, the spacer comprises a C₁ to C₂₀ (especially a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉ or C₂₀) straight, branched, saturated or unsaturated, substituted or non-substituted, alkyl, alkoxy or aromatic moiety, a polymer moiety such as a polyalkylene or polyalkylene glycol polymer containing 4 to 150 carbons, or a peptide linkage, each optionally additionally comprising at least one dendritic moiety. The spacer may be substituted with one or more N, S or O atoms.

Preferably, the polymer contains 4-130, especially 10-100, 2-80 or 40-60 carbons.

Preferably, the polyalkylene glycol is polyethylene glycol.

The photo-reactive group is preferably attached to the support via a spacer, the spacer being a C₁ to C₂₀ straight, branched, saturated or unsaturated, substituted or non-substituted, alkoxy or aromatic moiety, a polymer, such as a polyalkylene polymer containing 4 to 130, especially 10 to 100, 20 to 80 or 40 to 60 carbons, or a peptide linkage.

Preferably, the spacer is attached to the photo-reactive group via an ester, an ether, an amide, an amine, a thioether or a sulfone group. The inventors have identified that changing the ester or ether spacer allows the reactivity of the photo-reactive group to be modified so that it can react in a different manner with the molecule. This allows the activity of the photo-reactive group to be changed, thus allowing it to react with different parts of a ligand and present different parts of the ligand to the library.

Preferably, the spacer group is a polyalkylene glycol, such as polyethylene glycol. Preferably, the spacer group is a polymer polyalkylene, such as ethylene glycol, monomers.

The number of monomers, such as ethylene glycol, in the spacer is preferably 2 to 60. For example Tentagel™ comprises a core with polyethylene glycol molecules attached. The number of ethylene glycol monomers in such systems is typically 20 to 50. However, 2 to 10 or 10 to 20 may be used.

Preferably, the spacer group additionally comprises a dendritic moiety.

A dendritic moiety is one which contains a core moiety attached to which are a number of side groups, for example one or more additional dendritic groups, one part of the spacer connecting to the surface or support, or a further part of the spacer which comprises the photo-reactive group. That is, the dendritic group may be positioned within the spacer moiety with, for example, one or more carbon or other linking atoms proximal to the surface or support, and a second portion of the spacer distal to the support which contains attached to it a photo-reactive moiety. The proximal portion of the spacer serves to link the system to the surface or support, via the dendritic group and the distal part of the spacer moiety to one or more photo-reactive groups.

Alternatively, the dendritic group(s) may be connected directly to the support or surface via a functional group, with the spacer attached to another part of the dendritic group.

The dendritic moiety, in addition to the proximal part of the spacer moiety, may be attached to one or more photo-reactive groups. However, additionally one or more functional groups.

A plurality of dendritic moieties (typically 2 to 10, especially 3, 4, 5, 6, 7, 8 or 9) may be attached to one another to form a substantially tree-like structure. This allows a number of separate number of photo-reactive groups to be attached to a surface or support via a single proximal spacer attachment point.

The synthesis of dendrimers is known (Grayson (2001), Chem. Technol. Biotechnol., Vol. 76, page 903). The synthesis of polyamine dendrimers with sequentially added monomers containing nitrile groups, which were reduced to amines to allow the subsequent generation of dendrimers to be synthesised has been demonstrated (Buhleier E., et al. (1978) Synthesis. Page 155). Other dendrimers known in the art include Starburst™ oligomers which are grown from a core of ammonia with subsequent diamine branches added (Tomalia D. A. (1986) Macromol., Vol. 19, page 2466). Arborols which contain an aromatic core with Tris (hydroxymethyl)aminomethane as the branch in point have also been prepared (Newkome G. R. (1986), J. Am. Chem. Soc., Vol. 108, page 849). Furthermore, more recently dendrimers containing a phosphorous sulphur core with alkyl and aromatic branches have been prepared by Hawker C. J. and Fréchet J. M. J. (1990) J. Am. Chem. Soc., Vol. 112, page 7638). The use of amine groups as dendritic groups has been demonstrated in Basso A., et al. (Tet. Lett. 2000, page 3763).

Preferably, the dendritic group is selected from an amine group and an arborol.

However, most preferably the dendritic moiety is a triazine group. The Inventors have found that triazine groups allow the synthesis of the surfaces and supports of the invention via relatively easy or controllable synthetic routes. Preferably the triazine is a 1, 3, 5 triazine. The spacer comprising the photo-reactive group may be linked meta or para to the part of the triazine group linked directly or indirectly via a proximal spacer moiety, to the surface or support.

The triazine may be conveniently attached to the support by, for example, an amine group, or alternatively connected to the support via one or more spacer end groups, as defined below, or alternatively via one or more linking carbon atoms in combination with such spacer end groups or amine groups. The triazine may be attached directly or indirectly via one or more carbon atoms and a linking group such as an ester, amine or ether group and/or another group formed by reacting the end group with the support or surface to form a covalent bond with the surface or support.

Preferably, the spacer is connected to the surface or support (optionally via one or more dendritic groups as defined above) via a spacer end group selected from methyl, —SH, —CO₂H, —CONH₂, —NH₂, —OH, —CHO, —OC(O)CH₂CH₂ and —OC(O)C(CH₃)CH₂.

Preferably, the photo-reactive group is attached to the support via a dendritic group. The dendritic group preferably comprises attached thereto, optionally via a spacer, at least one further photo-reactive group and/or a second functional group. A dendritic group is one which contains a core moiety attached to which are a number of side groups, for example one or more photo-reactive groups (optionally attached to the dendritic group via a spacer), or alternatively one or more additional functional groups. Preferably the dendritic group comprises a triazine branch point.

The additional functional groups may be a protein resistant group.

Hence the method of the invention preferably utilises a support comprising a protein resistant group. The protein resistant groups may be attached via a dendritic group, directly onto the support or via a spacer to the support. The function of a protein resistant group is to prevent, or reduce, non-specific binding by the peptides or proteins expressed on the bacteriophage or alternatively by e.g. the bacteriophage coat proteins itself to the support. Preferred protein resistant groups include betaines, polyethylene glycol, taurine and derivatives thereof.

Protein-resistant surfaces are discussed in the article by Kane R. S., et al. (Langmuir 2003, Vol. 19, pages 2388-2391). Other protein resistant surfaces include mannitol dimethylacetamide, and poly(ethyleneimine) DMSO, HMPA and derivatives thereof. The compounds may be derivatised by reacting with hexamethyl-phosphoramide. Examples of such groups include:
-(EG)₆OH
—O(Man)
—C(O)N(CH₃)CH₂(CH(OCH₃))₄CH₂OCH₃
—N(CH₃)₃⁺Cl⁻/HS(CH₂)₁₁SO₃⁻Na (1:1)
—N(CH₃)₂⁺CH₂CH₂SO₃⁻
—C(O)Pip(NAc)
—N(CH₃)₂⁺CH₂CO₂⁻
—O(Malt)
—C(O)(N(CH₃)CH₂C(O))₃N(CH₃)₂
—N(CH₃)₂⁺CH₂CH₂CH₂SO₃⁻
—C(O)N(CH₃)CH₂CH₂N(CH₃)P(O)(N(CH₃)₂)₂
—(S(O)CH₂CH₂CH₂)₃S(O)CH₃
   where
   Man=mannitol
   Malt=[Glc-α(1,4)-Glc-β(1)-]
   EG=ethylene glycol

Preferably the support and ligand are in an aqueous medium and are not evaporated to dryness prior to reacting the photoreactive group with the ligand. This ensures that water is structured around the protein resistant surface to optimise its protein resistant properties.

Preferably, the dendritic group is a triazine. The triazine may be conveniently attached to the support by, for example, an amine group. Photo-reactive groups may be attached to one or more of the meta- or para-positions on the triazine moiety.

Other dendritic groups include polyesters, polyamides, polycarbonates and polyurethanes.

Preferably, the photo-reactive group produces as an intermediate upon photo activation an intermediate selected from: a nitrene, a carbene, a free radical, a carbon electrophile.

Preferably, the photo-reactive group is selected from an arylazide, a purineazide, a pyrimidineazide, an acylazide, a diazoketone, a diazirine, a benzophenone, an enone, a dioxane, nitrobenzene, a diazonium salt and a phosphonium salt.

Preferably, the photo-reactive group is selected from phenylazide, a halo-substituted arylazide, a nitro-substituted arylazide, an imino-substituted arylazide or an acyl-substituted arylazide, an adenosinylazide, an azidoguanosine, an alkylazide, p-nitrobenzoyl azide, a triazole, a diazoacetate (such as farnesyl diazoacetate), a diazirine, benzophenone, an enone, a sulphur containing compound such as nitrobenzyl nitrobenzylmercaptopurine, thymidine, thioguanosine or thiouridine, a halogenated substrate (such as a dioxane 5-bromouridine), nitrobenzene, an aryldiazonium salt, a substituted or a non-substituted analogue thereof.

Preferably, the photo-reactive group is selected from: derivatives of diazirine, nitrobenzene, phenylazide, benzophenone and especially trifluoromethyldiazirine. the derivatives may be halo derivatives such as fluoro, chloro, bromo or iodo substituted and/or C₁ to C₅ alkyl, especially methyl, ethyl substituted propyl or butyl.

Most preferably, the photo-reactive groups are selected from:

### Ether or Ester Derivatives of:

3-(3-(trifluoromethyl)-3/H/-diazirin-3-yl)phenol
   or ester derivatives of:
4-(3-(trifluoromethyl)-3/H/-diazirin-3-yl)benzoic acid.

Preferably, the support comprises glass, silica, polystyrene or polyamide. Such supports are known in the art. They may be derivatised, for example with the addition of amine or hydroxyl groups to allow the attachment of the photo-reactive group, spacer or dendritic group.

The dendritic group may itself be attached to the support via a separate spacer group. The spacer group may be as defined above.

Preferably, the support is a bead or microbead, or a microtitre plate. The microtitre plates may be used in the 36 well or 96 well format and are preferably made of polystyrene. The sizes of microtitre plates are usually standardised in the art. The advantage of using a microtitre plate is that it allows it to be used within, for example, robotic applications without much modification to existing robotic systems for mass screening programs. The bead may be magnetic to allow the bead to be isolated using a magnet. Such beads are known per se under the trademark “Dynabead”. Dynabeads™ are available from Invitrogen Ltd. They comprise magnetic particles of, for example, iron, and a polymer coating. Magnetic beads are also available from other sources, such as Bioclone Inc., San Diego, Calif., USA.

The beads or microbeads may alternatively themselves be used with a microtitre plate by putting a measured dose in a microtitre well. In this latter format the microtitre plate may not be a support and may not comprise a photoreactive group as defined above.

Preferably, the support may be coated with, for example, an additional polymer such as polyethylene glycol. Commercially-available polystyrene beads coated with polyethylene glycol are known in the art. Indeed, they are manufactured under the trade name “TentaGel” (a trade mark of Rapp Polymere GmbH) and Novagel. TentaGel resins are available with a number of different functional groups attached. This allows a variety of chemistry to be used to attach the photo-reactive group, for example via the spacer and/or the dendritic group. Alternatively, a polystyrene plug, for example one manufactured under the trade mark “Synphase Lanterns” (obtainable from Mimotopes Pty Ltd.) may be used.

A further aspect of the invention provides a supported photo-reactive compound for use in a method of the invention comprising a photo-reactive group attached to a support via a spacer and a dendritic group, the dendritic group comprising attached thereto (optionally via a spacer), at least one further photo-reactive group and/or a second functional group.

Preferably, the second functional group is a protein resistant group, such as defined above.

A still further aspect of the invention provides a supported photo-reactive compound for use in the method according to the invention comprising a photo-reactive group attached to a support and a protein resistant group attached to a support.

Supports, photo-reactive groups and protein resistant group for any aspects of the invention may be as defined above.

A further aspect of the invention provides a compound for use in a method according to the invention comprising:

a magnetic bead support attached to a substituted or non-substituted benzophenone, optionally via a spacer group. The magnetic bead, benzophenones and spacers may be as defined above. The beads may additionally comprise one or more protein resistant groups as defined above.

Preferably, the components of the photo-reactive compound, such as the spacer, dendritic group, spacers, support and protein protecting groups are as defined above.

Preferably, the supported photo-reactive compound contains two different photo-reactive groups, or photo-reactive groups capable of reacting differently to each other, on the same support. For example, the same reactive moiety, but with a different ester or ether link to the spacer may be used to modify the activity of the photo-reactive group. Alternatively, two or more separate supports with different photoreactive groups may be provided.

A kit for use in methods of identifying proteins or peptides capable of binding a peptide comprising a supported photo-reactive compound as defined above are also provided. The kit preferably contains two or more different photo-reactive compounds with different photo-reactive groups, or alternatively the same photo-reactive group modified so that it reacts differently. The kit may additionally comprise an expression library as defined above, comprising a plurality of members, each member expressing a different peptide or protein. In particular, the kit preferably contains a microtitre plate as a support. The kit may additionally comprise instructions for using the kit in a method according to the invention.

The invention will now be described by way of example only, with reference to the following figures and examples:

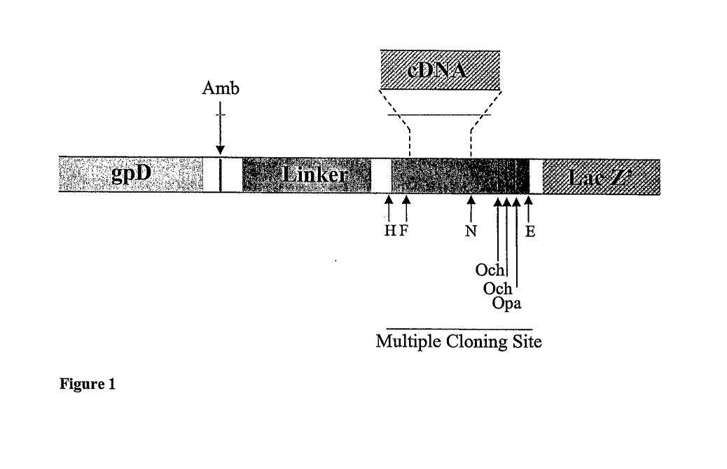

FIG. 1. Schematic representation of the gpD region in λfooDcSTOP. The fusion construct includes the sequences encoding the surface protein gpD, an amber stop codon (Amb), a polypeptide spacer (Linker), and a multiple cloning site. The multiple cloning site includes the sequences between restriction sites HindIII (H) and EcoRI (E), and cDNA inserts are cloned between the FseI (F) and NotI (N) sites. The cloning site also contains three stop codons (2× ochre, Och; 1× opal, Opa) each in a different reading frame. In a host that suppresses the amber stop codon gpD, the linker, and the cDNA clone are translated into a single protein molecule. The stop codons in the cloning site prevent the β-galactosidase gene (Lac Z′) downstream of the cDNA clone being included in the fusion protein.

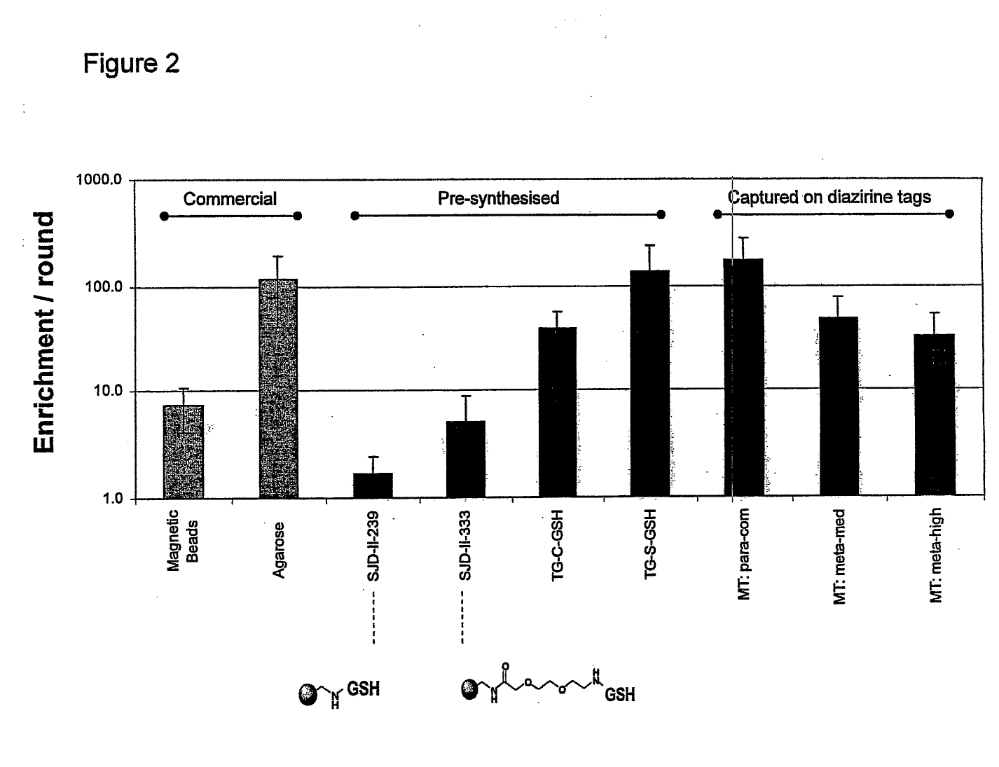

FIG. 2. Enrichment data of lambda phage expressing GST screened against various supported glutathione.

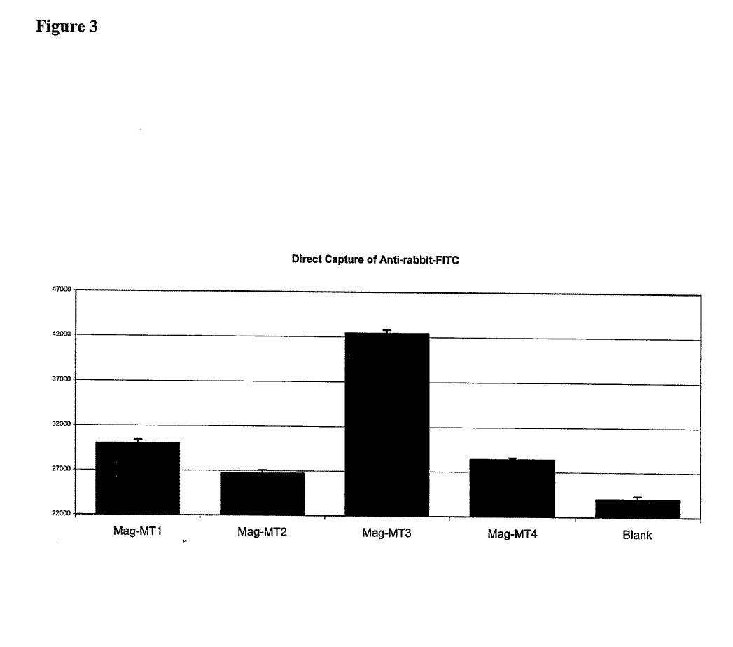

FIG. 3 shows the attachment of magnetic beads to anti-rabbit antibody labelled with a fluorescent label (FITC); MagMT1 (diazine label), MagMT2 (4-hydroxybenzophenone), MagMT3 (4-amino benzophenone) and a 1:1 mixture of MagMT2 and MagMT3 (shown as MagMT4). MagMT3 is shown to bind the antibody better than MagMT-1 or MagMT2 using daylight.

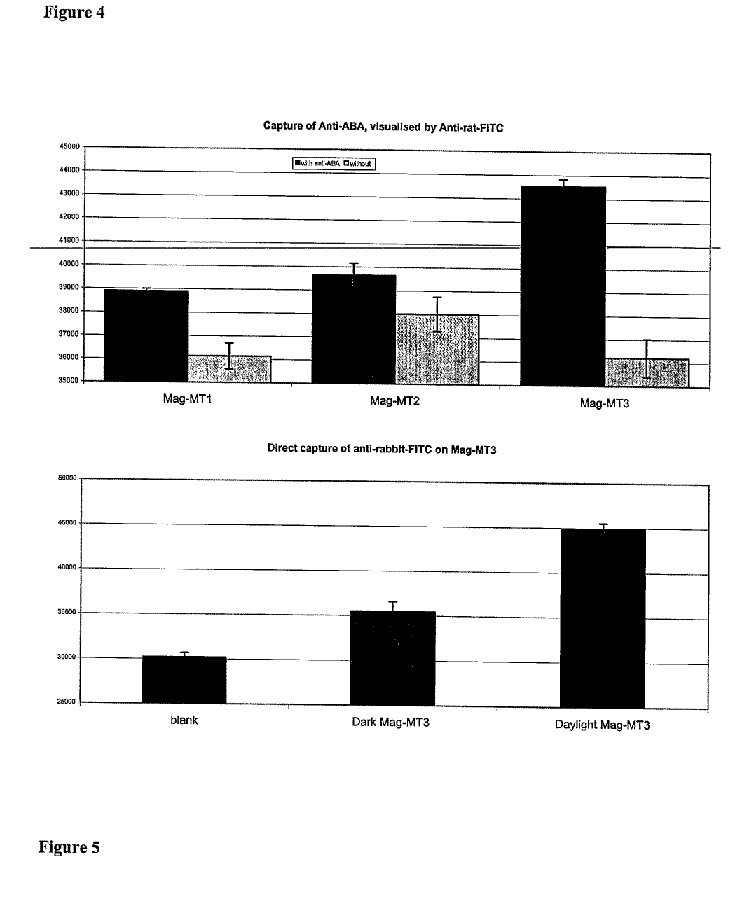

FIG. 4 shows the binding of MagMT1, MagMT2 and MagMT3 to rat anti-abscisic acid (anti-aba) antibody. The binding of the antibody was visualised with FITC-labelled anti-rat antibody. Dark blocks show binding of the anti-rat antibody to anti-aba bound to the beads. Light blocks show staining without anti-aba present. MagMT3 binds the anti-aba antibody better than MagMT2 and MagMT1. It also had lower background staining with anti-rat FITC than MagMT-2.

FIG. 5 shows the effect of exposing anti-rabbit-FITC antibody to MagMT3. Blank, no MagMT3; Dark Mag MT3 shows that some reaction occurred whilst the MagMT3 and antibody were being mixed due to some light leakage and exposure; Daylight MagMT3 shows full light exposure considerably increases the binding of MagMT3 to the antibody.

### PHOTO-REACTIVE GROUPS

### N-trifluoroacetylpiperidine 1

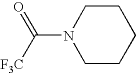 [0117] Nassal, M. Liebigs Ann. Chem. 1983, 1510.

A solution of piperidine (2.0 g, 24 mmol) and triethylamine (2.0 g, 20 mmol) in diethyl ether (50 ml) was cooled to 0° C. Trifluoroacetic anhydride (4.4 g, 20 mmol) was added dropwise over 15 min. The reaction was allowed to reach room temperature and stirred for a further 2 h. Aqueous hydrochloric acid (5 ml, 0.1 M) was introduced into the reaction and the mixture extracted with diethyl ether. The organic phase was dried over magnesium sulphate and evaporated under reduced pressure. The residue was purified by column chromatography (silica, DCM) to yield N-trifluoroacetylpiperidine colourless oil (3.0 g, 83%). νₘₐₓ: 2946, 2862, 1687, 1467, 1447, 1191, 1141, 1128 cm⁻¹. δ_{H} (300 MHz, CDCl₃): 1.57-1.61 (m, 6H); 3.46 (t, J=5, 2H); 3.54 (t, J=5, 2H) ppm. δ_{C} (75 MHz, CDCl₃): 24.5, 25.7, 26.7, 44.9, 47.2, 118.9, 155.4 ppm.

### 2,2,2-Trifluoro-1-(3-methoxyphenyl)ethanone 2

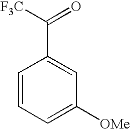 [0120] Hatanaka, Y; Hashimoto, M; Kurihara, H; Nakayama, H.; Kanaoka, Y. J. Org. Chem. 1994, 59, 383.

To a slurry of magnesium (1.4 g, 58 mmol) in THF (100 ml) was added 3-bromoanisole (6.7 g, 36 mmol). The mixture was allowed to spontaneously reflux for 1 h. A solution of N-trifluoroacetylpiperidine (5.8 g, 32 mmol) in THF (10 ml) was added and the reaction stirred at room temperature for 3 h. Saturated aqueous ammonium chloride solution (20 ml) was introduced into the reaction and the mixture extracted with diethyl ether. The organic phase was dried over magnesium sulphate and evaporated under reduced pressure. The residue was purified by column chromatography (silica, 2:1 petroleum ether: ethyl acetate) to give 2,2,2-trifluoro-1-(3-methoxyphenyl)ethanone as a pale orange oil (4.6 g, 73%). νₘₐₓ: 2937, 2828, 1715, 1598, 1582, 1249, 1198, 1136, 993, 959, 752, 734 cm⁻¹. δ_{H} (300 MHz, CDCl₃): 3.86 (s, 3H); 7.22 (ddd J=1, 3, 8, 1H); 7.43 (t J=8, 1H); 7.55 (s, 1H); 7.64 (d J=8, 1H) ppm. δ_{C} (75 MHz, CDCl₃): 55.8, 114.2, 115.6, 118.9, 122.6, 123.0, 130.5, 131.4, 160.3 ppm. δ_{F} (300 MHz, CDCl₃): −72.3 ppm.

### 2,2,2-Trifluoro-1-(3-methoxyphenyl)ethanone oxime 3

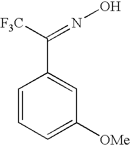 [0123] Hatanaka, Y; Hashimoto, M; Kurihara, H; Nakayama, H.; Kanaoka, Y. J. Org. Chem. 1994, 59, 383.

To a solution of 2,2,2-trifluoro-1-(3-methoxyphenyl)ethanone (6.2 g, 30 mmol) in ethanol (40 ml) and pyridine (40 ml) was added hydroxylamine hydrochloride (2.2 g, 31 mmol). The reaction was then heated to 60° C. for 20 h. The solvents were removed by evaporation and the residue suspended in diethyl ether (50 ml). The solution was washed with hydrochloric acid (1M, 3×200 ml) and extracted with further diethyl ether (3×100 ml). The combined organic phases were dried over magnesium sulphate and evaporated under reduced pressure. The residue was purified by column chromatography (silica, 5% methanol in DCM) to yield 2,2,2-trifluoro-1-(3-methoxyphenyl)ethanone oxime as a colourless oil (5.5 g, 83%). νₘₐₓ: 3312, 1705, 1603, 1581, 1247, 1189, 1128, 966, 733 cm⁻¹. δ_{H} (300 MHz, CDCl₃): 3.79 (s, 3H); 7.00 (ddd J=1, 3, 8, 1H); 7.06 (s, 1H); 7.07 (d J=8, 1H); 7.37 (t J=8, 1H); 9.88 (s, 1H) ppm. δ_{C} (75 MHz, CDCl₃): 55.8, 114.3, 116.6, 119.2, 121.2, 122.8, 127.6, 130.0, 159.8 ppm. δ_{F} (300 MHz, CDCl₃): −66.8 ppm. m/z: 136 (86%), 219 (45%).

### 2,2,2-Trifluoro-1-(3-methoxyphenyl)ethanone oxime tosylate 4

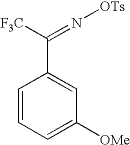 [0126] Hatanaka, Y; Hashimoto, M; Kurihara, H; Nakayama, H.; Kanaoka, Y. J. Org. Chem. 1994, 59, 383.

A solution of 2,2,2-trifluoro-1-(3-methoxyphenyl)ethanone oxime (5.5 g, 25 mmol) in DCM (80 ml) was cooled to 0° C. The solution was treated sequentially with tosyl chloride (6.9 g, 36 mmol), 4-dimethylamino pyridine (1.5 mmol, 0.18 g) and triethylamine (7.6 g, 75 mmol). The reaction was allowed to reach room temperature and stirred for a further 1 h. Water was introduced into the reaction and the mixture extracted with DCM. The organic phase was dried over magnesium sulphate and evaporated under reduced pressure to yield 2,2,2-trifluoro-1-(3-methoxyphenyl)ethanone oxime tosylate as a dark solid (9.0 g, 96%), which was used without further purification. δ_{H} (300 MHz, CDCl₃): 2.47 (s, 3H); 3.80 (s, 1H); 6.88 (s, 1H); 6.94 (d J=8, 1H); 7.04 (ddd J=1, 3, 8, 1H); 7.37 (d J=8, 1H); 7.38 (t J=8, 1H) ppm. δ_{C} (75 MHz, CDCl₃): 22.2, 55.8, 114.3, 117.8, 121.0, 121.7, 125.9, 126.3, 129.5, 130.5, 131.4, 140.2, 146.7, 159.9 ppm. δ_{F} (300 MHz, CDCl₃): −67.3 ppm.

### 3-(3-Methoxyphenyl)-3-(trifluoromethyl)diaziridine 5

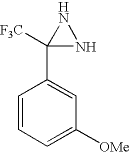 [0129] Hatanaka, Y; Hashimoto, M; Kurihara, H; Nakayama, H.; Kanaoka, Y. J. Org. Chem. 1994, 59, 383.

A solution of 2,2,2-trifluoro-1-(3-methoxyphenyl)ethanone oxime tosylate (5.8 g, 16 mmol) in DCM (100 ml) was cooled to −78° C. Ammonia gas (˜20 ml) was introduced and condensed into the solution using a dry-ice trap. The reaction was allowed to reflux at −78° C. for 8 h, then allowed to reach room temperature overnight, by which time the ammonia had evaporated out of the reaction vessel. The residue was washed with water and extracted into DCM. The organic phase was dried with magnesium sulphate and evaporated under reduced pressure to yield 3-(3-methoxyphenyl)-3-(trifluoromethyl)diaziridine as a colourless oil (2.9 g, 86%). νₘₐₓ: 3255, 2941, 1604, 1586, 1245, 1215, 1139, 715, 693 cm⁻¹. δ_{H} (300 MHz, CDCl₃): 2.21 (d J=9, 1H, NH); 2.72 (d J=9, 1H, NH); 3.72 (s, 3H); 6.87 (ddd J=1, 3, 8, 1H); 7.05 (s, 1H); 7.10 (d J=8, 1H); 7.23 (t J=8) ppm. δ_{C} (75 MHz, CDCl₃): 55.0, 113.4, 115.5, 120.1, 121.5, 125.2, 129.5, 132.8, 159.4 ppm. δ_{F} (300 MHz, CDCl₃): −76.6 ppm. m/z: 123 (40%), 172 (25%), 189 (55%), 204 (35%), 219 (45%).

### 3-(3-Methoxyphenyl)-3-(trifluoromethyl)-3H-diazirine 6

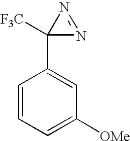

### Dark Procedure: Darkened Fumehood, all Flasks/Beakers Foil Coated, Amber NMR Tube.

A solution of 3-(3-methoxyphenyl)-3-(trifluoromethyl)diaziridine (0.2 g, 0.9 mmol) in diethyl ether (10 ml) was treated with manganese dioxide (1.6 g, 18 mmol). The reaction was stirred vigorously for 2 h. The manganese was removed by filtration through cellite, washed with diethyl ether (100 ml). The organic was dried over magnesium sulphate and evaporated under reduced pressure to yield 3-(3-methoxyphenyl)-3-(trifluoromethyl)-3H-diazirine as a colourless oil (0.18 g, 91%). δ_{H} (300 MHz, CDCl₃): 3.81 (s, 3H); 6.68 (s, 1H); 6.77 (d J=8, 1H); 6.94 (d J=8, 1H); 7.31 (t J=8, 1H) ppm. δ_{C} (75 Mhz, CDCl₃): 55.7, 112.6, 115.8, 118.4, 119.1, 130.4, 166.24 ppm. δ_{F}. (300 MHz, CDCl₃): −66.3 ppm.

### 3-(3-(Trifluoromethyl)-3H-diazirin-3-yl)phenol 7

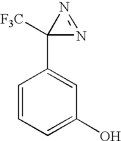 [0134] Hatanaka, Y; Hashimoto, M; Kurihara, H; Nakayama, H.; Kanaoka, Y. J. Org. Chem. 1994, 59, 383.

### Dark Procedure: Darkened Fumehood, all Flasks/Beakers Foil Coated, Amber NMR Tube.

A solution of 3-(3-methoxyphenyl)-3-(trifluoromethyl)-3H-diazirine (0.42 g, 1.9 mmol) in DCM was cooled to 0° C. Boron tribromide (1M in DCM, 3.9 mmol) was added dropwise. The reaction was maintained at 0° C. for a further 5 h. Water was introduced into the reaction and the mixture was extracted with DCM. The organic phase was dried over magnesium sulphate and evaporated under reduced pressure to yield 3-(3-(trifluoromethyl)-3H-diazirin-3-yl)phenol as an oil (0.38 g, 97%). νₘₐₓ: 3367, 1672, 1610, 1587, 1264, 1152, 731, 691 cm⁻¹. δ_{H} (300 Mhz, CDCl₃): 4.84 (s, 1H, OH); 6.66 (s, 1H); 6.71 (d J=8, 1H); 6.87 (dd, J=2, 8, 1H); 7.25 (t, J=8, 1H) ppm. δ_{F} (300 Mhz, CDCl₃): −67.9 ppm.

### Ether linked polymer supported 3-(3-(trifluoromethyl)-3H-diazirin-3-yl)phenol 8

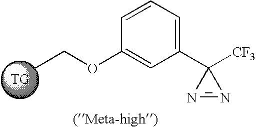

### Dark Procedure: Darkened Fumehood, all Flasks/Beakers Foil Coated.

To a slurry of Tentagel S—OH (0.25 g, 0.25 mmol/g) in THF (10 ml) was added 343-(trifluoromethyl)-3H-diazirin-3-yl)phenol (38 mg, 0.19 mmol). The mixture was then treated with triphenylphosphine (81 mg, 0.31 mmol) and diethylazodicarboxylate (54 mg, 0.31 mmol). The reaction was shaken for 24 h. νₘₐₓ: 2864, 1742, 1716, 1602, 1452, 1296, 1248, 1094, 700 cm⁻¹.

Absorbances at 1602 and 1296 cm⁻¹ demonstrate presence of correct product. 1742 and 1716 cm⁻¹ demonstrate incorporation of diethylazodiacetate DEAD by-products. Further washing does not reduce this contamination, so it was concluded that some light activation must have occurred to allow the tag to ‘pick up’ the DEAD.

### Ester linked polymer supported 3-(3-(trifluoromethyl)-3H-diazirin-3-yl)phenol 9

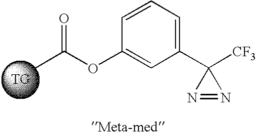

### Dark Procedure: Darkened Fumehood, all Flasks/Beakers Foil Coated.

To a slurry of Tentagel S—COOH (0.25 g, 0.25 mmol/g) was added 3-(3-(trifluoromethyl)-3H-diazirin-3-yl)phenol (40 mg, 0.2 mmol). The mixture was then treated with EDCI (0.2 mmol, 40 mg) and 4-dimethylaminopyridine (0.7 mg, 6 μmol). The reaction was shaken for 24 h. The resin was removed by filtration and washed with DCM, methanol, 1:1 DCM/methanol, acetone, and DCM. The resin was then dried under vacuum at 50° C. νₘₐₓ: 2867, 1762, 1696, 1655, 1602, 1452, 1348, 1094, 947, 700 cm⁻¹.

### Polymer supported 4-(3-(Trifluoromethyl)-3H-diazirin-3-yl)benzoic acid 10

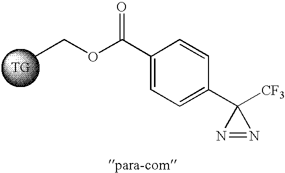

### Dark Procedure: Darkened Fumehood, all Flasks/Beakers Foil Coated.

Tentagel S—OH (0.2 g, 0.25 mmol/g) was added to a solution of 4-(3-(trifluoromethyl)-3H-diazirin-3-yl)benzoic acid (23 mg, 0.1 mmol) in DMF (10 ml). EDCI (0.1 mmol, 19 mg) and 4-dimethylaminopyridine (0.6 mg, 5 μmol) were added and the reaction shaken for 24 h. The resin was removed by filtration and washed with DCM, methanol, 1:1 DCM/methanol, acetone, and DCM. The resin was then dried under vacuum at 50° C. νₘₐₓ: 2864, 1721, 1294, 1095, 700 cm⁻¹.

### Definitions of Magic Tag Codes for Biological Data.

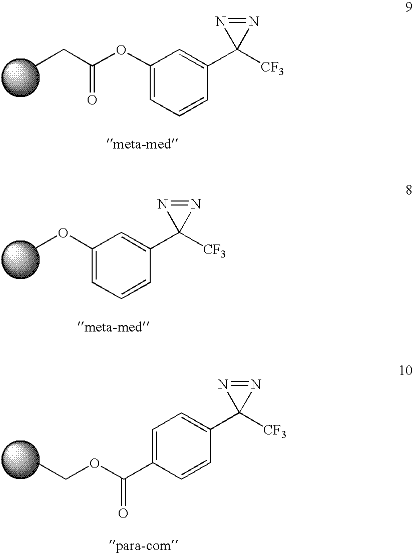

### Alternative Branched Chain Dendritic Group Formation:

Basso, A.; Evands, B.; Pegg, N.; Bradley, M. Tetrehedron Lett. 2000, 3763.

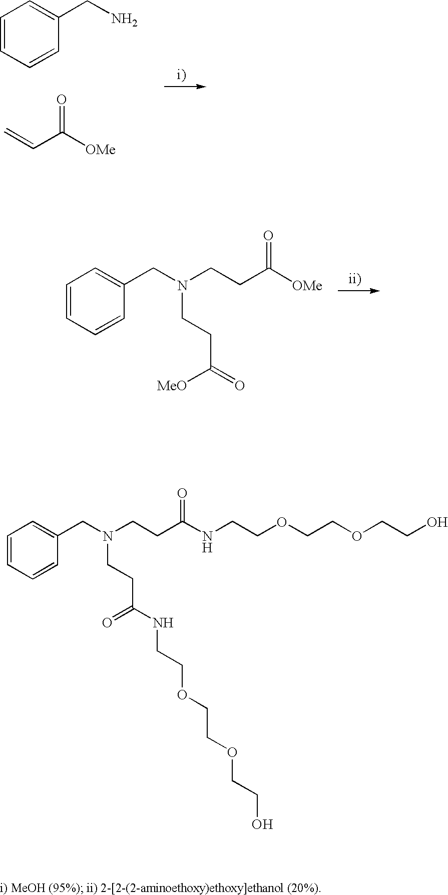

Basso, A.; Bands, B.; Pegg, N.; Bradley, M. Tetrehedron Lett. 2000, 3763.

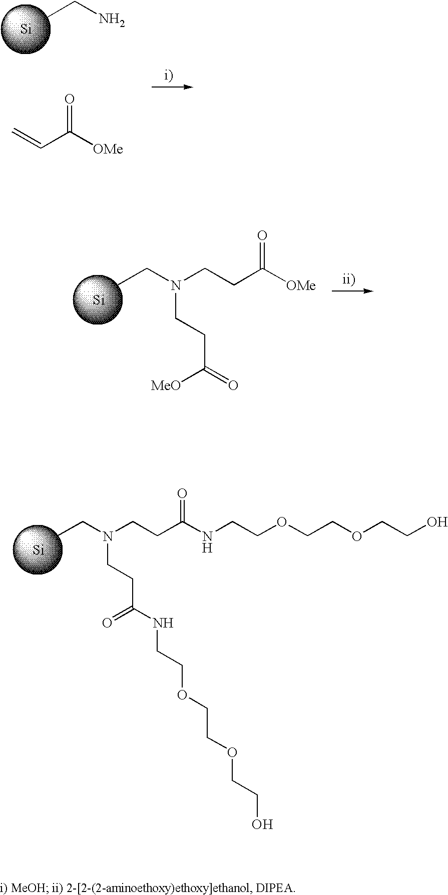

### S-Linked Polymer Supported Glutathione Synthesis

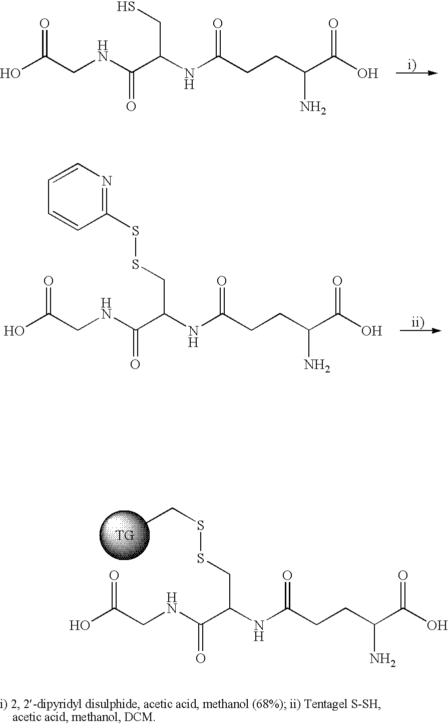

### 2-pyridyl glutathione disulphide 11

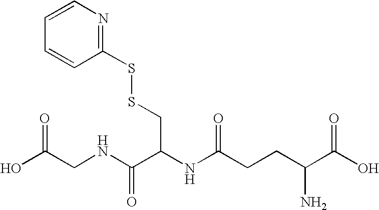

A mixture of glutathione (0.5 g, 1.6 mmol) and 2,2′ dipyridyl disulphide (0.7 g, 3.3 mmol) was suspended in methanol (10 ml). Acetic acid (0.13 g, 2.2 mmol) was added and the solution stirred vigorously for 3 days. The yellow precipitate was removed and triturated with chloroform. The product was then dried overnight under vacuum at 50° C. to yield 2-pyridyl glutathione disulphide as a yellow solid (0.36 g, 68%). νₘₐₓ: 3348, 3045, 1672, 1638, 1508, 1415, 1228, 1113, 1079 cm⁻¹. δ_{H} (300 MHz, D₂O): 1.90-1.99 (m, 2H); 2.27-2.34 (m, 2H); 2.98 (dd J=10, 14, 1H); 3.24 (dd J=4, 14); 3.63 (t J=7, 1H); 3.78 (s, 2H); 4.46 (dd J=4, 10, 1H); 7.27 (td J=2, 5, 1H); 7.74-7.83 (m, 2H); 8.31 (d J=5, 1H) ppm. δ_{C} (75 MHz, D₂O): 26.3, 31.5, 39.6, 42.0, 53.0, 54.1, 120.1, 122.9, 122.9, 128.2, 140.2, 148.5, 163.6, 164.2, 174.9, 175.0 ppm. m/z: 232 (20%), 417 (15%).

### Tentagel glutathione disulphide 12

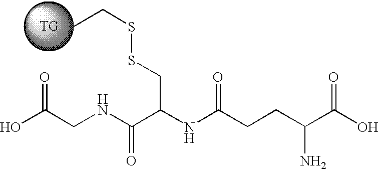

A swollen sample of Tentagel S—SH (1.5 g, 0.25 mmol/g) in DCM (10 ml). A solution of acetic acid (0.5 mmol, 30 mg) in methanol (10 ml) was added, followed by 2-pyridyl glutathione disulphide (0.32 g, 0.8 mmol). The reaction was shaken for 7 days. The resin was removed and washed successively with: DCM, 1:1 DCM/methanol, water, acetone and DCM. The resin was then dried under vacuum at 50° C. for 24 h. νₘₐₓ: 3500, 2866, 1668, 1654, 1452, 1093, 1030, 700 cm⁻¹. Anal. C, 63.08; H, 8.43; N, 0.96%.

Loading calculated from percentage N, 0.23 mmol/g. Maximum theoretical loading: 0.23 mmol/g.

### C-Linked Polymer Supported Glutathione Series Synthesis:

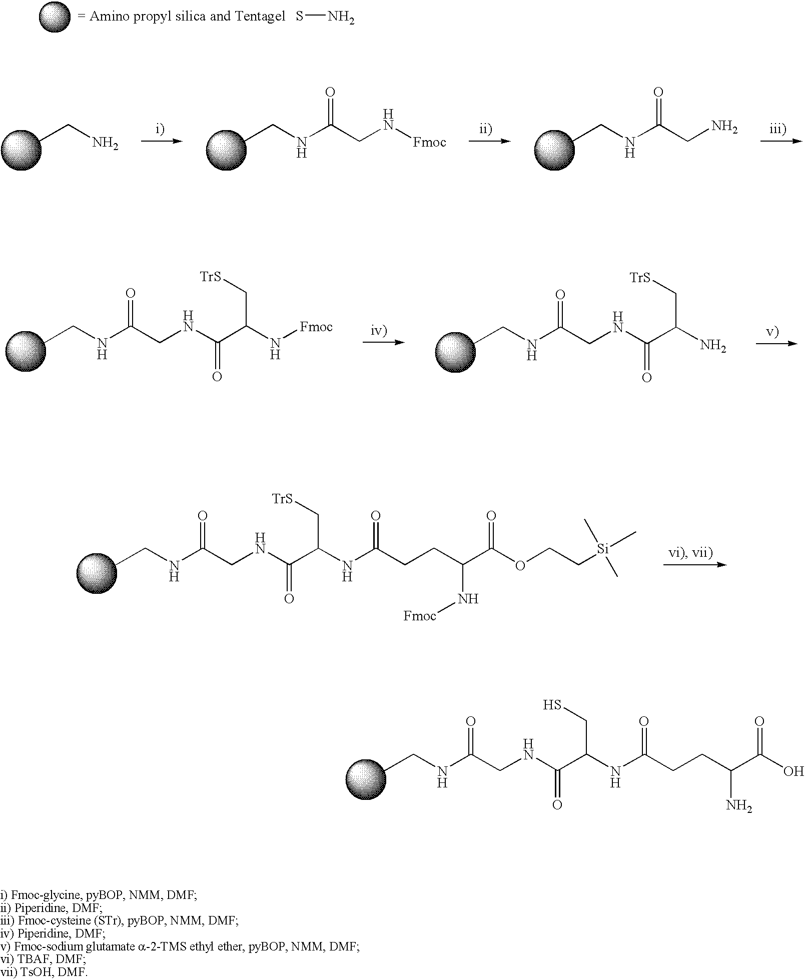

### Polymer supported Fmoc-glycine 13

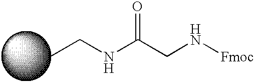

### Tentagel 13a

Tentagel S—NH₂ (2.0 g, 0.25 mmol/g) was added to a solution of Fmoc-glycine (0.3 g, 1.0 mmol) and pyBOP (0.52 g, 1.0 mmol) in DMF (10 ml). N-methylmorpholine (1.0 mmol, 0.1 g) was added and the reaction shaken for 4 h. The resin was removed by filtration and washed successively with: DMF, DCM, 1:1 DCM/methanol, DCM. The resin was then dried under vacuum at 50° C. for 24 h. νₘₐₓ: 3025, 2864, 1723, 1673, 1651, 1451, 1344, 1095, 700 cm⁻¹. Anal: C, 64.77; H, 8.46; N, 0.58%.

Loading calculated from percentage N, 0.21 mmol/g. Maximum theoretical loading: 0.23 mmol/g.

### Silica 13b

Aminopropyl silica (0.5 g, 1.4 mmol/g) was added to a solution of Fmoc-glycine (0.42 g, 1.4 mmol) and pyBOP (0.7 g, 1.4 mmol) in DMF (10 ml). N-methylmorpholine (0.2 g, 1.4 mmol) was added and the reaction shaken for 4 h. The resin was removed by filtration and washed successively with: DMF, DCM, 1:1 DCM/methanol, DCM. The resin was then dried under vacuum at 50° C. for 24 h. νₘₐₓ: 3291, 2938, 1704, 1652, 1538, 1039, 794 cm⁻¹. Anal: C, 15.34; H, 2.24; N, 2.89%.

Loading calculated from percentage N, 1.03 mmol/g. Maximum theoretical loading: 1.0 mmol/g.

### Polymer Supported Glycine 14

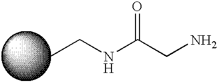

### Tentagel 14a

Tentagel supported Fmoc-glycine (2.0 g, 0.23 mmol/g) was shaken in a 20% solution of piperidine in DMF (10 ml) for 3 h. The resin was removed by filtration and washed successively with: DMF, DCM, 1:1 DCM/methanol, DCM. The resin was then dried under vacuum at 50° C. for 24 h. νₘₐₓ: 3059, 3026, 2866, 1672, 1653, 1451, 1347, 1092, 728, 699 cm⁻¹. Anal: C, 63.67; H, 8.65; N, 0.67%.

Loading calculated from percentage N, 0.24 mmol/g. Maximum theoretical loading: 0.24 mmol/g.

### Silica 14b

Silica supported Fmoc-glycine (0.6 g, 1.0 mmol/g) was shaken in a 20% solution of piperidine in DMF (10 ml) for 3 h. The resin was removed by filtration and washed successively with: DMF, DCM, 1:1 DCM/methanol, DCM. The resin was then dried under vacuum at 50° C. for 24 h. νₘₐₓ: 3316, 2966, 1652, 1047, 794 cm⁻¹. Anal: C, 7.26; H, 1.67; N, 2.71%.

Loading calculated from percentage N, 0.97 mmol/g. Maximum theoretical loading: 1.3 mmol/g.

### Polymer supported gly-Fmoc-cys (STr) 15

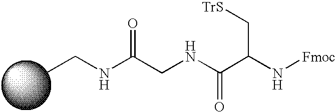

### Tentagel 15a

Tentagel supported glycine (1.85 g, 0.24 mmol/g) was added to a solution of Fmoc-cysteine (STr) (0.51 g, 0.88 mmol) and pyBOP (0.46 g, 0.88 mmol) in DMF (10 ml). N-methylmorpholine (0.88 mmol, 89 mg) was added and the reaction shaken for 4 h. The resin was removed by filtration and washed successively with: DMF, DCM, 1:1 DCM/methanol, DCM. The resin was then dried under vacuum at 50° C. for 24 h. νₘₐₓ: 3060, 3026, 2864, 1716, 1665, 1651, 1451, 1347, 1090, 700 cm⁻¹. Anal: C, 62.89; H, 8.34; N, 0.79%.

Loading calculated from percentage N, 0.19 mmol/g. Maximum theoretical loading: 0.21 mmol/g.

### Silica 15b

Silica supported glycine (0.5 g, 1.3 mmol/g) was added to a solution of Fmoc-cysteine (STr) (0.8 g, 1.4 mmol) and pyBOP (0.7 g, 1.4 mmol) in DMF (10 ml). N-methylmorpholine (0.2 g, 1.4 mmol) was added and the reaction shaken for 4 h. The resin was removed by filtration and washed successively with: DMF, DCM, 1:1 DCM/methanol, DCM. The resin was then dried under vacuum at 50° C. for 24 h. νₘₐₓ: 3273, 2962, 1716, 1652, 1353, 1043, 791 cm⁻¹. Anal: C, 15.92; H, 2.09; N, 3.36%.

Loading calculated from percentage N, 0.80 mmol/g. Maximum theoretical loading: 0.74 mmol/g.

### Polymer supported gly-cys (STr) 16

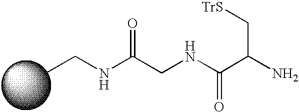

### Tentagel 16a

Tentagel supported gly-Fmoc-cys (STr) (1.8 g, 0.21 mmol/g) was shaken in a 20% solution of piperidine in DMF (10 ml) for 3 h. The resin was removed by filtration and washed successively with: DMF, DCM, 1:1 DCM/methanol, DCM. The resin was then dried under vacuum at 50° C. for 24 h. νₘₐₓ: 3062, 3023, 2864, 1660, 1645, 1451, 1347, 1091, 700 cm⁻¹. Anal: C, 63.01; H, 8.43; N, 0.90%.

Loading calculated from percentage N, 0.21 mmol/g. Maximum theoretical loading: 0.22 mmol/g.

### Silica 16b

Silica supported gly-Fmoc-cys (STr) (0.4 g, 0.74 mmol/g) was shaken in a 20% solution of piperidine in DMF (10 ml) for 3 h. The resin was removed by filtration and washed successively with: DMF, DCM, 1:1 DCM/methanol and DCM. The resin was then dried under vacuum at 50° C. for 24 h. Anal: C, 12.19; H, 1.94; N, 2.87%.

Loading calculated from percentage N, 0.68 mmol/g. Maximum theoretical loading: 0.88 mmol/g.

### Polymer Supported Protected Glutathione 17

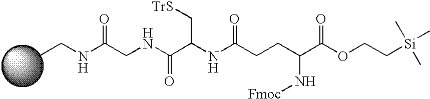

### Tentagel 17a

Tentagel supported gly-cys (STr) (1.6 g, 0.22 mmol/g) was added to a solution of Fmoc-sodium glutamate α-2-TMS ethyl ether (0.34 g, 0.7 mmol) and pyBOP (0.37 g, 0.7 mmol) in DMF (10 ml). N-methylmorpholine (70 mg, 0.7 mmol) was added and the reaction shaken for 4 h. The resin was removed by filtration and washed successively with: DMF, DCM, 1:1 DCM/methanol, DCM. The resin was then dried under vacuum at 50° C. for 24 h. νₘₐₓ: 2864, 1720, 1661, 1646, 1451, 1093, 840, 700 cm⁻¹. Anal: C, 62.18; H, 7.94; N, 0.27%.

### Silica 17b

Silica supported gly-cys (STr) (0.3 g, 0.88 mmol/g) was added to a solution of Fmoc-sodium glutamate α-2-TMS ethyl ether (0.25 g, 0.53 mmol) and pyBOP (00.28 g, 53 mmol) in DMF (10 ml). N-methylmorpholine (53 mg, 0.53 mmol) was added and the reaction shaken for 4 h. The resin was removed by filtration and washed successively with: DMF, DCM, 1:1 DCM/methanol, DCM. The resin was then dried under vacuum at 50° C. for 24 h. νₘₐₓ: 2949, 1715, 1652, 1539, 1044, 791 cm⁻¹. Anal: C, 16.28; H, 2.54; N, 3.28%.

Loading calculated from percentage N, 0.59 mmol/g. Maximum theoretical loading: 0.62 mmol/g.

### Polymer Supported Glutathione 18

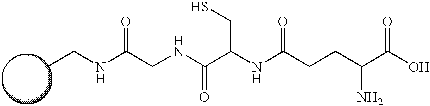

### Tentagel 18a

A suspension of tentagel supported protected glutathione (1.5 g, 0.2 mmol/g) in DMF (10 ml) was treated with tetra-n-butyl ammonium fluoride (1M in THF, 0.6 mmol). The reaction was shaken for 2 h. The resin was removed by filtration and washed successively with: DMF, DCM, 1:1 DCM/methanol and DCM. The resin was then dried under vacuum at 50° C. for 24 h. νₘₐₓ: 3507, 2960, 2901, 1660, 1649, 1451, 1406, 1393, 1080, 700 cm⁻¹. Anal: C, 61.88; H, 8.21; N, 0.98%.

Loading calculated from percentage N, 0.18 mmol/g. Maximum theoretical loading: 0.21 mmol/g.

The resin was then re-suspended in methanol (20 ml) and treated with p-toluene sulphonic acid (90 mg, 0.5 mmol). The mixture was shaken for 24 h. The resin was removed by filtration and washed successively with: DCM, 1:1 DCM/methanol and DCM. The resin was then dried under vacuum at 50° C. for 24 h. νₘₐₓ: 3332, 3025, 2863, 1731, 1670, 1451, 1347, 1094, 700 cm⁻¹. Anal: C, 64.05; H, 8.30; N, 1.14%

Loading calculated from percentage N, 0.20 mmol/g. Maximum theoretical loading: 0.23 mmol/g.

### Silica 18b

A suspension of silica supported protected glutathione (0.3 g, 0.62 mmol/g) in DMF (10 ml) was treated with tetra-n-butyl ammonium fluoride (1M in THF, 0.4 mmol). The reaction was shaken for 2 h. The resin was removed by filtration and washed successively with: DMF, DCM, 1:1 DCM/methanol and DCM. The resin was then dried under vacuum at 50° C. for 24 h. νₘₐₓ: 3279, 2967, 1664, 1652, 1540, 1046, 790 cm⁻¹. Anal: C, 16.79, 2.55, N, 3.21%.

Loading calculated from percentage N, 0.57 mmol/g. Maximum theoretical loading: 0.78 mmol/g.

The resin was then re-suspended in methanol (20 ml) and treated with p-toluene sulphonic acid (80 mg, 0.4 mmol). The mixture was shaken for 24 h. The resin was removed by filtration and washed successively with: DCM, 1:1 DCM/methanol and DCM. The resin was then dried under vacuum at 50° C. for 24 h. νₘₐₓ: 3062, 2683, 1666, 1646, 1536, 1046, 794 cm⁻¹. Anal: C, 17.64; H, 2.38; N, 2.63%.

Loading calculated from percentage N, 0.47 mmol/g. Maximum theoretical loading: 0.97 mmol/g.

Arabidopsis thaliana cDNA Domain Library

A library of 5,000,000 bacteriophage lambda (phage) particles was constructed. Each phage particle in the library can display on its surface a single domain of an Arabidopsis thaliana protein. Domains are displayed through fusion to the protein gpD that is located on the surface of the phage particle. A. thaliana proteins were fused to gpD by cloning fragments of cDNAs into a novel phage vector λfooDcSTOP (FIG. 1). In λfooDcSTOP cDNA is cloned downstream of the gene gpD in the multiple cloning site, and the two sequences are separated by an amber stop codon. When gpD is translated in an E. coli host that does not suppress the stop codon then little fusion protein is synthesised. However, when translated in an E. coli host that does suppress the stop codon then a lot of the fusion protein is synthesised. Subsequently, both the fusion protein and the native protein are incorporated into phage particles.

This library was constructed in four stages: 1. the phage vector λfooDc (Mikawa et al., 1996; obtained from Ichi Maruyama, Scripps Research Institute, La Jolla, Calif., U.S.A.) was modified to create the vector λfooDcSTOP; 2. cDNA fragments were prepared from A. thaliana tissue; 3. cDNA fragments were cloned into the novel phage vector and packaged into phage particles; 4. the library of phage particles was amplified. During the first stage, the sequence between the HindIII and EcoRI sites in λfooDc was replaced with a short sequence containing (from 5′-3′): a. two rare restriction sites FseI and NotI; b. three stop codons in each of the three different reading frames. The two restriction sites were introduced to force cDNA inserts to be cloned in one direction. The three stop codons were introduced to prevent the sequence downstream of the cDNA insert being translated as part of the fusion protein. During the second stage, mRNA was isolated from A. thaliana ecotype Landsberg erecta seedlings grown in liquid medium. Fragments of cDNA were then synthesised from this mRNA, using a random prime method. During the third stage, cDNA fragments were ligated between the FseI and NotI sites of λfooDcSTOP. Fragments of cDNA were used because whole proteins can be detrimental to the growth of phage, and protein domains often retain the biological activity that they exhibit when attached to the whole protein. In addition, fragments of cDNA were used to allow membrane proteins to be represented in the library. This is because when displayed on a phage particle the transmembrane domains are likely to prevent the inclusion of the fusion protein in phage particles. The ligated molecules were then packaged into phage particles, yielding a library of 5,000,000 independent clones. During the fourth stage, this library was amplified by propagating the phage on solid medium and recovering the amplified library using a liquid buffer.

The initial characterisation of the library indicates that it can be used to screen A. thaliana proteins for novel functions. In brief, the cDNA clones were amplified from 96 phage particles. The size of each clone was determined, and 20 of the clones were sequenced. These data indicate that most of the phage in the library contain a cDNA clone of at least 100 amino acids. Since domains of A. thaliana proteins are generally accepted to be around 100 amino acids, most phage in the library can display at least one domain. These size data also indicate that phage particles containing no cDNA clone, and that can therefore be detrimental to the application of the library, are rare. In order to demonstrate that phage particles in the library displayed domains of A. thaliana proteins on their surfaces, biopans (see biopan results section) were conducted against antibodies that bind specifically to plant proteins. In most of these biopans phage particles displaying a domain of the respective A. thaliana protein were affinity selected from the library.

### BIBLIOGRAPHY

Mikawa, Y. G., Maruyama, I. N., and Brenner, S. (1996). Surface Display of Proteins on Bacteriophage λ Heads. J. Mol. Biol., 262, 21-30.

### Protocol

This protocol is provided by way of example only. Other expression libraries known in the art may also be used.

### Biopan: λGST Against Reduced-Glutathione

PEG/NaCl 1 L

| | | |
|---|---|---|
| 26% w/v PEG 8000 | 260.0 | g |
| 2.6 M NaCl | 151.9 | g |
| dH₂O | to 1 | L |

After standard autoclave cycle, cool on ice to prevent precipitation. Store at RT (room temperature).

TBS 500 ml

| | | |
|---|---|---|
| 50 mM Tris-HCl pH7.5 | 3.30 | g |
| 150 mM NaCl | 4.38 | g |
| 10 mM MgSO₄ | 1.23 | g (MgSO₄•7H₂O) |
| dH₂O | to 500 | ml |

Autoclave and store at RT.

TBS 0.1% Tween 20 (500 ml)

| | |
|---|---|
| 1 x TBS | 500 ml |
| 0.1% Tween 20 | 0.5 ml |

Autoclave and store at RT.

TBS 0.5% Tween 20 (500 ml)

| | |
|---|---|
| 1 x TBS | 500 ml |
| 0.5% Tween 20 | 2.5 ml |

Autoclave and store at RT.

Elution Buffer

| | | |
|---|---|---|
| 1 x TBS 0.5% Tween 20 | 1 | ml |
| 20 mM reduced glutathione (GSH) | 6.1 | mg |
| pH ~7.3 | 12-15 | ul 1 M Tris-HCl pH 9.1 |

SM 1 L

| | | |
|---|---|---|
| 10 mM NaCl | 5.8 | g |
| ~8 mM MgSO₄•7H₂O | 2.0 | g |
| 50 mM Tris-HCl | 50 | ml 1 M Tris-HCl pH 7.5 |
| 0.1% Gelatin | 5 | ml 2% w/v Gelatin |

to 1000 ml with ddH₂O. Autoclave and store at RT.

Binding Buffer

TBS 0.1% Tween 20
5% non-fat milk powder
0.25% BSA
(Brenner 1994) +
E. coli 4° C. Stocks

Grow E. coli overnight in NZCYM (37° C., 250 rpm, 250 ml flask). Transfer into a 50 ml tube and centrifuge for 10 min at 7,000×g, room temperature. Discard supernatant and resuspend in ˜35 ml of 10 mM MgSO₄. Ensure the OD₆₀₀ₙₘ is 2.0. Store at 4° C. (use by end of 14 days).

| | For | For |
|---|---|---|
| NZCYM | 1000 ml | 900 ml: |
| 1.0% NZ amine (N-Z-Amine A, Sigma C0626) | 10 g | 9 g |
| ~9 mM NaCl (BDH) | 5 g | 4.5 g |
| 0.5% bacto-yeast extract (yeast extract, Sigma Y1625) | 5 g | 4.5 g |
| 0.1% casamino acids (Hy-CaseAmino, Sigma C0501) | 1 g | 0.9 g |
| ~8 mM MgSO₄•7H₂O (Sigma) | 2 g | 1.8 g |
| ~1% Agar (BDH) | 11 g | 9.9 g |

to desired volume with ddH₂O
pH 7.0 with concentrated HCl

Add agar for [NZCYM AGAR]. Autoclave and store at RT

### Day 1: Preparation of Phage

Add 1 ml E. coli strain TG1 (OD₆₀₀ 2.0) to 50 ml NZCYM in a 250 ml flask. Grow at 37° C. with shaking at 250 rpm, until OD₆₀₀ 0.5 (usually around 2 hours).

Be careful not to allow the OD₆₀₀ to exceed 0.5 as some strains of E. coli grow faster than others. For instance, while E. coli strain Q526 usually takes around 2 hours to reach this OD₆₀₀, TG1 can take less than 2 hours. Hence, other strains of E. coli may be used.
2. Add 10⁹ phage (pfu) to the log-phase culture. Continue incubation at 37° C. with shaking at 250 rpm for around 4 hours (cell lysate should be visible after this time or sooner). Where possible, prepare different phage separately (for example λfooDcSTOP_{(GST)} and λfooDcSTOP_{(no insert)}).
3. Make the following additions to each flask, in the following order:
   1 ml Aristar Chloroform
   50 ul 10 mg/ml RNase A, in 1×TE buffer (SIGMA R4875)) Combine these two and make a 25 ul 20 mg/ml DNase I, in 1×TE buffer (SIGMA DN-25)) single addition of 75 ul
   Then, incubate for a final 10 min at 37° C., with shaking at 250 rpm.
4. Decant as much of the liquid as possible into a 30 ml centrifuge tube (try and avoid large clumps of cell lysate). Then centrifuge at 16,000×g for 10 min, 4° C.
5. Decant 30 ml of the supernatant into a fresh 30 ml centrifuge tube, containing a pre-chilled 10 ml aliquot of PEG/NaCl. Mix and chill on ice for 1 hour with regular manual inversion.
6. Centrifuge at 16,000×g for 15 min, 4° C.
7. Remove supernatant and invert on a paper towel to remove any residual. Re-suspend phage pellet in 1 ml SM (or Binding Buffer), to which 7% DMSO should be added prior to storage at −80° C.
8. Titre the phage stock using the 10 ul Spot method, and store at −80° C.

### Day 2: Biopan

For each biopan add the solid support to a 1.5 ml Eppendorf tube (20 ul Novogen GST•Mag™ 71084/biopan; 100 ul 50% slurry Sigma Glutathione-Agarose G—4510 or 20 mg exemplified supports).
2. Centrifuge for ˜30 sec at 13,000 rpm (microcentrifuge), then remove storage buffers from commercial supports *. * When only using magnetic beads, beads are collected using a magnetic holder for 1 min.
3. Add 1 ml TBS 0.1% Tween 20 and re-suspend the beads, ensuring that re-suspension is visibly complete. Then wash the supports using numerous manual inversions (at least 10, with gentle vibration). Centrifuge for ˜30 sec at 13,000 rpm (microcentrifuge), then remove supernatant. This is the wash procedure.
4. Wash supports 3 times in 1 ml TBS 0.1% Tween 20 (i.e. repeat the previous step, 3 more times).
5. Wash supports in 1 ml of Binding Buffer (TBS, 0.1% Tween 20, 5% Milk, 0.25% BSA).
6. Pre-block by adding 1 ml Binding Buffer and tumbling at RT for 1 hour.
7. Add 10¹⁰ phage in 1 ml of Binding Buffer, and tumble at RT for 1 hour. This is the Input (abbreviated, Ip). For a control pan, use a mixture of 10¹⁰ λfooDcSTOP_{(library)} and 10⁴-10⁷λfooDcSTOP_{(GST)}. Store an aliquot of this mixture at −80° C. for confirmation of λfooDcSTOP_{(GST)} frequency using Western blot (add 7% DMSO and 0.1% gelatin for storage at −80° C.).
8. Centrifuge for −30 sec at 13,000 rpm (microcentrifuge), then discard the supernatants from each biopan.
9. Wash supports 5 times in 1 ml TBS 0.5% v/v Tween 20 (abbreviated, W 1-5). Collect final wash, titre the wash, and store at −80° C. (add 7% DMSO for storage at −80° C.). When multiple pans are being conducted be careful not to cross contaminate as this may effect the titres of the washes. If comparing the titres of the final wash and eluate then allow 1 min for the final wash only.
10. Add 1 ml Elution Buffer (TBS, 0.5% v/v Tween 20, 20 mM GSH, pH ˜7.3), and manually invert at RT for 1 minute. See below for how to prepare the Elution Buffer. This is the eluate (abbreviated, E)
11. Store the eluate on ice and titre (10 ul Spot method)+. + If analysing the eluate this is the end of the entire protocol. For storage at −80° C. add 7% DMSO and 0.1% gelatin.
12. Grow E. coli Q526 in 50 ml NZCYM (37° C., 250 rpm, 250 ml flask) to OD₆₀₀ 0.5. Then add the remaining eluate and incubate at 37° C. overnight with shaking (250 rpm).

### Day 3: Non-Selective Amplification of the Eluate

Make the following additions to each flask, in the following order:
1 ml Aristar Chloroform
50 ul 10 mg/ml RNase A, in 1×TE buffer (SIGMA R4875)) Combine these two and make a
25 ul 20 mg/ml DNase I, in 1×TE buffer (SIGMA DN-25)) single addition of 75 ul
Then, incubate for a final 10 min at 37° C., with shaking at 250 rpm.
2. Decant as much of the liquid as possible into a 30 ml centrifuge tube (try and avoid large clumps of cell lysate). Then centrifuge at 16,000×g for 10 min, 4° C.
3. Decant 30 ml of the supernatant into a fresh 30 ml centrifuge tube, containing a pre-chilled 10 ml aliquot of PEG/NaCl. Mix and chill on ice for 1 hour with regular manual inversion.
4. Centrifuge at 4 16,000×g for 15 min, 4° C.
5. Remove supernatant and invert on a paper towel to remove any residual. Re-suspend phage pellet in 1 ml SM, to which 7% DMSO should be added prior to storage at −80° C.
6. Titre the phage stock/prep (10 ul Spot method), and store at −80° C. This is the Output (abbreviated, Op).
7. Use the output to prepare the Input for subsequent rounds of panning.
   Measuring Phage Titre (pfu/ml): the 10 μl Spot Method

Prepare a dilution series of the phage solution in SM. Use a 1/10 series for the input phage and 1/100 series for all other samples.
2. Plate 30 ul of selected dilutions:

Prepare a petri dish (plate) containing a lawn of E. coli. For each set of four dilutions mix 200 ul of E. coli stock with 3 ml NZCYM 0.7% agarose (50° C.) and plate on ˜20-30 ml 37° C. NZCYM Agar (top agar). For best results ensure NZCYM agar plates are dry (allow 40 min in flow cabinet and overnight at RT), and following the addition of the top agar allow 0.5 hours until the lid of the dish is replaced.
ii) Add spots of selected dilutions. Ensure dilutions are well suspended and then plate three 10 ul spots of each. 12 spots fit comfortably on one plate (8.5 cm diameter).
3. Following overnight (15-16 hours) incubation at 37° C., count pfu against a gloss-black background. Plates can be wrapped in cling-film to prevent excessive drying.
4, When counting the pfu, follow these rules:

The maximum number of pfu/10 ul spot which can be counted accurately has been empirically determined to be 50. A weighted mean is used to calculate the titre.

### Plaque-Lifts Followed by Western Blot Detection

1) Prepare a dilution series of the following phage suspensions, using SM buffer:
Biopan output
Biopan input
Lambda GST (positive control)
Library (negative control)
   2) Plate selected dilutions * from 1) as follows: * For the output and input select a range of dilutions such that at least one will contain a number of Lambda GST in 100 ul that can be scored accurately (>10, <300). For the two controls only one dilution is needed and it should contain 100-1000 pfu in 100 ul.

Combine 100 ul of each dilution with 200 ul TG1 E. coli stock (incubate at 37° C.; 20 min).

Combine the mixture from i) with 3 ml NZCYM 0.7% agarose (50° C.) and plate on ˜20-30 ml 37° C. NZCYM Agar (9 cm diameter petri dish). For best results ensure NZCYM agar plates are dry (allow 40 min in flow cabinet and overnight at RT), and following the addition of the NZCYM 0.7% agarose allow 0.5 hours until the lid of the dish is replaced.

Grow overnight at 37° C.

3) Count the number of plaques on each plate, using a black background. Select at least one plate containing each of the different suspensions. For the input and output from the biopan this plate should contain an accurately scorable number of Lambda GST plaques.
4) For each plate cut nitrocellulose membrane (Amersham, Hybond ECL) to shape, label, and moisten in 1×PBS buffer.
5) Draw excess moisture from membrane, then transfer to the surface of an NZCYM AGAR plate. Allow 30-60 minutes at RT for effective transfer of protein. Try not to move the membrane after touching the plate and be sure to remove bubbles of air.
6) Remove membrane from the plate, dry at RT (protein side up, on 3M Whatman filter paper), and store in filter paper indefinitely.
7) Detect the plaques containing Lambda GST by processing each membrane as follows (all steps performed at room temperature):
i. Immerse the membrane in 10 ml PBS pH 7.3 and swirl for 10 min.
ii. Immerse the membrane in 10 ml PBS 3% (w/v) BSA 2% (w/v) dried milk powder and swirl for 60 min.
iii. Immerse the membrane in 10 ml PBS 3% (w/v) BSA 1/10,000 Goat anti-GST (Amersham 27-4577-01) and swirl for 60 min.
iv. Immerse the membrane in 15 ml PBS 0.1% (v/v) Tween 20 and swirl for 5 min. Repeat this wash with a further 15 ml PBS 0.1% (v/v) Tween 20. Immerse the membrane in 15 ml PBS 0.5% (v/v) Tween 20 1 M NaCl and swirl for 5 minutes. Repeat this wash with a further 15 ml PBS 0.5% (v/v) Tween 20 1 M NaCl.
v. Immerse the membrane in 10 ml PBS 3% (w/v) BSA 1/5,000 Rabbit anti-Goat (Sigma A-3540) and swirl for 60 min.
vi. Immerse the membrane in 15 ml PBS 0.1% (v/v) Tween 20 and swirl for 5 min. Repeat this wash with a further 15 ml PBS 0.1% (v/v) Tween 20. Immerse the membrane in 15 ml PBS 0.5% (v/v) Tween 20 1 M NaCl and swirl for 10 minutes. Immerse the membrane in 15 ml PBS 0.1% (v/v) Tween 20 and swirl for 5 min. Repeat this wash twice, each with a further 15 ml PBS 0.1% (v/v) Tween 20. Immerse the membrane in 15 ml PBS and swirl for 5 min.
vii. Develop blot using ECL kit (ECL Western Blotting Detection Reagents Amersham Pharmacia Biotech RPN 2209) in accordance with manufacturer's instructions. Expose to photographic film for 1 min. Repeat and adjust time of exposure accordingly.
viii. Count plaque-like signals on a white-light source (GST positive plaques). Use this number to estimate the frequency of phage displaying GST in the eluate. Use this frequency to estimate the enrichment that occurred during biopanning.

### Stock of X10 Phosphate Buffered Saline (PBS) pH 7.3

Total volume 5 l.

| | |
|---|---|
| NaCl | 400 g |
| KCl | 10 g |
| Na₂HPO₄ | 57.5 g |
| KH₂PO₄ | 10 g |

Tween 20—Polyoxyethylene sorbitan monolaurate Sigma P-1379
BSA—Bovine serum albumin Sigma A-4503

### Results

FIG. 2 shows the enrichment data obtained for a number of different supported glutathione.

The magnetic beads (Dynabeads™, Dynal Corp—available from Invitrogen Corp). Agarose supported glutathione is commercially available from Sigma Ltd.

The supports indicated by the prefix “SJD” are silica-based supports with amino propyl attached substituents. Initial data indicates that these may be used to enrich and isolate bacteriophage with GST. It is expected that better results may be obtained using longer spacer groups to leave the ligand (GSH) more open to access by the phage.

Better results were obtained using Tentagel with the long PEG spacer groups.

TG-C-GSH was attached chemically via the C-terminus using established peptide chemistry. TG-S-GSH had glutathione attached chemically via a the sulphur atom using a disulphide linkage through displacement of a mixed disulphide intermediate. This shows that orientation of glutathione affects the amount of enrichment observed.

However, comparative amounts of enrichment or better can be obtained using the photoreactive supports of the invention (prefixed “MT”). The difference between the supports refers to the para or meta positions of the photoreactive group on the dendritic group on the support.

### Further Work on Magnetic Bead Supports

### Magnetic bead supported 2-(2-[2-acetimidoethoxy]ethoxy)chloride

Magnetic beads (1 ml, 50 mg) in a 1.5 ml tube were captured using a magnetic stand. The supernatant was decanted off, and the beads treated with phosphate-buffered saline (PBS) (1 ml). After brief vortexing, the beads were again captured and the supernatant removed. A solution of 2-(2-[2-chloroethoxy]ethoxy)acetic acid (61 mg, 0.3 mmol), EDCI (70 mg, 0.3 mmol) and DMAP (5 mg, 0.04 mmol) in PBS (1 ml) and DMF (0.1 ml) was added to the washed beads. The reaction was shaken for 24 h, then the beads were successively washed, captured and the supernatant removed with PBS (×3), DMF (×3) and methanol (×3). Finally, PBS (1 ml) was added for storage.

### Magnetic bead supported 2-(2-[2-acetimidoethoxy]ethoxy)phthalimide

Magnetic beads (1 ml, 50 mg) in a 1.5 ml tube were captured using a magnetic stand. The supernatant was decanted off, and the beads treated with phosphate-buffered saline (PBS) (1 ml). After brief vortexing, the beads were again captured and the supernatant removed. A solution of 2-(2-[2-phthalimidoethoxy]ethoxy)acetic acid (50 mg, 0.2 mmol), EDCI (70 mg, 0.3 mmol) and DMAP (5 mg, 0.04 mmol) in PBS (1 ml) and DMF (0.1 ml) was added to the washed beads. The reaction was shaken for 24 h, then the beads were successively washed, captured and the supernatant removed with PBS (×3), DMF (×3) and methanol (×3). Finally, PBS (1 ml) was added for storage.

### Magnetic bead supported 2-(2-[2-acetimidoethoxy]ethoxy)amine

Magnetic bead supported 2-(2-[2-acetimidoethoxy]ethoxy)phthalimide (1 ml, 50 mg) in a 1.5 ml tube were captured using a magnetic stand. The supernatant was decanted off, and the beads treated with ethanol (1 ml). After brief vortexing, the beads were again captured and the supernatant removed. A solution of hydrazine monohydrate (0.1 ml, 0.2 mmol) in water (0.1 ml) and ethanol (1 ml) was added to the washed beads. The reaction was vortexed briefly, then shaken for 3 h. The beads were successively washed, captured and the supernatant removed with PBS (×3) and ethanol (×3). Finally, PBS (1 ml) was added for storage.

### Magnetic bead supported 2-(2-[2-acetimidoethoxy]ethoxy)acetimidopropanoic acid

Magnetic bead supported 2-(2-[2-acetimidoethoxy]ethoxy)amine (1 ml, 50 mg) in a 1.5 ml tube were captured using a magnetic stand. The supernatant was decanted off, and the beads treated with acetonitrile (1 ml). After brief vortexing, the beads were again captured and the supernatant removed. A solution of succinic anhydride (13.2 mg, 0.15 mmol) and diisopropylethylamine (0.02 ml, 0.05 mmol) in acetonitrile (1 ml) was added to the washed beads. The reaction was vortexed briefly, then shaken for 3 h. The beads were successively washed, captured and the supernatant removed with PBS (×3) and ethanol (×3). Finally, PBS (1 ml) was added for storage.

### Magnetic bead supported 3-(3-acetoxyphenyl)-3-(trifluoromethyl)-3H-diazirine MagMT1

### Dark Procedures Followed to Minimise Exposure to Light.

Magnetic bead supported 2-(2-[2-acetimidoethoxy]ethoxy)acetimidopropanoic acid (1 ml, 50 mg) in a 1.5 ml tube were captured using a magnetic stand. The supernatant was decanted off, and the beads treated with a solution of 3-(3-hydroxyphenyl)-3-(trifluoromethyl)-3H-diazirine (25 mg, 0.1 mmol), EDCI (20 mg, 0.1 mmol) and DMAP (5 mg, 0.04 mmol) in PBS (1 ml) and DMF (0.5 ml). The reaction was vortexed briefly, then shaken for 24 h. The beads were successively washed, captured and the supernatant removed with PBS (×3) and ethanol (×3). Finally, PBS (1 ml) was added for storage.

### Magnetic bead supported 4-hydroxybenzophenone MagMT2

Sodium (1 mg) was added to ethanol (1 ml). On cessation of visible reaction, 4-hydroxybenzophenone (0.02 g, 0.1 mmol) was added. Meanwhile, Magnetic bead supported 2-(2-[2-acetimidoethoxy]ethoxy)chloride (1 ml, 50 mg) in a 1.5 ml tube were captured using a magnetic stand. The supernatant was decanted off, and the beads treated with ethanol (1 ml). After brief vortexing, the beads were again captured and the supernatant removed. The bright yellow ethanol solution was then added to the washed beads. The reaction was vortexed briefly, then shaken for 3 h. The beads were successively washed, captured and the supernatant removed with PBS (×3) and ethanol (×3). Finally, PBS (1 ml) was added for storage.

### Magnetic bead supported 4-amidobenzophenone MagMT3

Magnetic bead supported 2-(2-[2-acetimidoethoxy]ethoxy)amine (1 ml, 50 mg) in a 1.5 ml tube were captured using a magnetic stand. The supernatant was decanted off, and the beads treated with a solution of 4-benzoylbenzoic acid (35 mg, 0.15 mmol), EDCI (60 mg, 0.3 mmol) and DMAP (10 mg, 0.08 mmol) in PBS (1 ml) and DMF (0.4 ml). The reaction was vortexed briefly, then shaken for 3 h. The beads were successively washed, captured and the supernatant removed with PBS (×3) and ethanol (×3). Finally, PBS (1 ml) was added for storage.

### General Procedure for Immobilisation

Magnetic beads (0.1 ml, 5 mg beads in PBS) were combined with a solution of protein (0.2 ml, 2 μg in PBS) in a black tube, and vortexed briefly. The contents of the tube were transferred to a clear tube, and the tube exposed to daylight for 10 minutes. The beads were then captured and washed with PBS (×6) and then assayed, either directly by fluorescence, or indirectly via a secondary fluorescent antibody (following a BSA blocking step).

To demonstrate that magnetic beads could be made to bind to proteins, or indeed peptides, the beads were reacted with fluorescently labelled anti-rabbit FITC antibody and binding detected directly by measuring the preset fluorescently labelled antibody. The results are shown in FIGS. 3 and 5. MagMT4 is a 1:1 mix of MagMT2 and MagMT3. Alternatively, the magnetic beads were reacted with rat anti-abscisic acid antibody and visualised with FITC-labelled anti-rat antibody. The results are shown in FIG. 4.

The results show that benzophenones, such as 4-amidobenzophenone, can advantageously bind proteins or peptides, prior to screening with the expression library.

The use of Magnetic beads allows phage bound to the magnetic beads to be easily separated from solution.

1. A method of identifying a peptide or protein capable of binding a ligand which comprises:
(i) providing a support, the support comprising a photoreactive group;
(ii) reacting the photoreactive group with a ligand to attach the ligand to the support and produce a supported ligand;
(iii) providing an expression library comprising a plurality of members, each member expressing a different peptide or protein;
(iv) screening the expression library to identify one or more peptides or proteins which bind to the supported ligand;
(v) isolating the member or each member of the library which expresses a peptide or protein which binds to the ligand; and
(vi) identifying the peptide or protein which binds to the ligand.

2. A method according to claim 1, wherein 2 or more photoreactive groups are provided either bound onto the same support or alternatively to separate supports.

3. Method according to claim 1, wherein the peptide or protein identified in step (vi) is (a) sequenced to establish the amino acid sequence of the peptide or protein or (b) a portion of the member of the expression library encoding the peptide or protein is sequenced to identify a nucleotide sequence encoding the peptide or protein.

4. Method according to claim 1, wherein the photo-reactive group is attached to the support via a spacer, the spacer being a C₁ to C₂₀ straight, branched, saturated or unsaturated, substituted or non-substituted, alkoxy or aromatic moiety, a polymer, such as a polyalkylene polymer containing 4 to 130 carbons, or a peptide linkage.

5. Method according to claim 4, wherein the spacer is attached to the photo-reactive group via an ester, an ether, an amide, an amine, a thioether or a sulfone group.

6. Method according to claim 4, wherein the spacer group is a polyethylene glycol.

7. A method according to claim 1, wherein the photo-reactive group is attached to the support via a dendritic group, the dendritic group comprising attached thereto, optionally via a spacer, at least one further photo-reactive group and/or a second functional group.

8. A method according to claim 1, wherein the support comprises a protein resistant group.

9. A method according to claim 8, wherein the protein protecting group is attached to the support via the dendritic group, as defined in claim 7.

10. Method according to claim 8, wherein the dendritic group contains a triazine branching point.

11. A method according to claim 8, wherein the protein resistant group is selected from a polyethylene glycol, a betaine, taurine and derivatives thereof.

12. A method according to claim 1, wherein the photo-reactive group produces as an intermediate upon photo-activation an intermediate selected from: a nitrene, a carbene, a free radical, a carbon electrophile.

13. A method according to claim 12, wherein the photo-reactive group is selected from an aryl azide, a purine azide, a pyrimidine azide, an acyl azide, a diazoketone, a diazirine, a benzophenone, an enone, a dioxane, nitrobenzene, a diazonium salt and a phosphonium salt.

14. A method according to claim 12, wherein the photo-reactive group is selected from a diazirine, nitrobenzene, phenylazide and benzophenone.

15. A method according to claim 1, wherein the support comprises glass, silica, polystyrene and polyamide.

16. A method according to claim 1, wherein the support is a bead or a microtitre plate.

17. Method according to claim 1 comprising providing two different photo-reactive groups, each photo-reactive group attached to the same or a different support, and reacting a plurality of the ligands with the photo-reactive groups prior to step (iii).

18. Method according to claim 1 wherein the expression library is selected from a phage display library, a bacterial cell surface display library, a yeast cell surface display library and a baculovirus insect expression library.

19. Method according to claim 1, wherein the expression library is a cDNA library.

20. A supported photo-reactive compound for use in a method according to claim 1 comprising a photo-reactive group attached to a support via a spacer and a dendritic group, the dendritic group comprising attached thereto, optionally via a spacer, at least one further photo-reactive group and/or a second functional group.

21. A compound according to claim 20, wherein the second functional group is a protein resistant group.

22. A compound according to claim 20, wherein the dendritic group contains a triazine branching point.

23. A supported photo-reactive compound for use in a method according to claim 1, comprising a photo-reactive group attached to a support and a protein resistant group attached to the support.

24. A compound according to claim 23, wherein the photo-reactive group is attached via a spacer group.

25. A compound according to claim 21, wherein the protein resistant group is selected from polyethylene glycol, betaine, taurine and derivatives thereof.

26. A compound according to claim 20, wherein the spacer is selected from a C₁ to C₂₀ straight, branched, saturated or unsaturated, substituted or non-substituted, alkoxy or aromatic moiety.

27. A compound according to claim 26, wherein the spacer is attached to the photo-reactive group via an ester or an ether group.

28. A compound according to claim 26, wherein the spacer group is polyethylene glycol.

29. A compound according to claim 20, wherein the photo-reactive group produces as an intermediate upon photo activation an intermediate selected from: a nitrene, a carbene, a free radical, a carbon electrophile.

30. A compound according to claim 29, wherein the photo-reactive group is selected from an aryl azide, a purine azide, a pyrimidine azide, an acyl azide, a diazoketone, a diazirine, a benzophenone, an enone, a dioxane, nitrobenzene, a diazonium salt and a phosphonium salt.

31. A compound according to claim 29, wherein the photo-reactive group is selected from an aryl azide, a purine azide, a pyrimidine azide, an acyl azide, a diazoketone, a diazirine, a benzophenone, an enone, a dioxane, nitrobenzene, a diazonium salt and a phosphonium salt.

32. A compound according to claim 20, wherein the support comprises glass, silica, polystyrene and polyamide.

33. A compound according to claim 32, wherein the support is a bead or a microtitre plate.

34. A compound according to claim 33, wherein the support is a magnetic bead.

35. A compound according to claim 23, wherein the photoreactive group is a substituted or non-substituted benzophenone.

36. A compound for use in a method according to claim 1 comprising a magnetic bead support attached to a photo-reactive substituted or non-substituted benzophenone group, optionally via a spacer group.

37. A compound according to claim 20, wherein two or more different supported photo-reactive groups are provided as the support.

38. A kit for use in identifying a peptide or a protein capable of binding a ligand comprising a supported photo-reactive compound as defined in claim 20.

39. A kit according to claim 38, wherein two or more different photo-reactive compounds with different photo-reactive groups are provided on separate supports or the same support.

40. A kit according to claim 38, additionally comprising an expression library comprising a plurality of members, each member expressing a different peptide or protein.

41. A kit according to claim 38, wherein the support is a microtitre plate.

42. A kit according to claim 38, wherein the support is a magnetic bead.

43. A kit according to claim 38, wherein the kit additionally comprises instructions for using the kit.

44. A compound according to claim 23, wherein the protein resistant group is selected from polyethylene glycol, betaine, taurine and derivatives thereof.

45. A compound according to claim 23, wherein the spacer is selected from a C₁ to C₂₀ straight, branched, saturated or unsaturated, substituted or non-substituted, alkoxy or aromatic moiety.

46. A compound according to claim 23, wherein the photo-reactive group produces as an intermediate upon photo activation an intermediate selected from: a nitrene, a carbene, a free radical, a carbon electrophile.

47. A compound according to claim 23, wherein the support comprises glass, silica, polystyrene and polyamide.

48. A compound according claim 23, wherein two or more different supported photo-reactive groups are provided as the support.

49. A compound according to claim 36, wherein two or more different supported photo-reactive groups are provided as the support.

50. A kit for use in identifying a peptide or a protein capable of binding a ligand comprising a supported photo-reactive compound as defined in claim 23.

51. A kit for use in identifying a peptide or a protein capable of binding a ligand comprising a supported photo-reactive compound as defined in claim 36.
